(19) United States
(12) Patent Application Publication
(10) Pub. No.: US 2015/0299744 A1
(43) Pub. Date: Oct. 22, 2015

(54) METHODS FOR OBTAINING A GENETICALLY MODIFIED PLANT OR MICROBE AND FOR INCREASING OIL YIELD

(71) Applicant: SIME DARBY MALAYSIA BERHAD, Kuala Lumpur (MY)

(72) Inventors: Jaime Yoke Sum Low, Selangor Darul Ehsan (MY); Nurliyana Binti Ruzlan, Selangor Darul Ehsan (MY); Wilonita Win, Selangor Darul Ehsan (MY); Chin Ming Lim, Selangor Darul Ehsan (MY); Noor Azizah Binti Musa, Selangor Darul Ehsan (MY); Yick Ching Wong, Selangor Darul Ehsan (MY); Huey FAng Teh, Selangor Darul Ehsan (MY); David Ross Appleton, Selangor Darul Ehsan (MY); Hirzun Bin Mohd Yusof@Hassan, Selangor Darul Ehsan (MY); Harikrishna A/L Kulaveerasingam, Selangor Darul Ehsan (MY)

(21) Appl. No.: 14/436,051

(22) PCT Filed: Apr. 1, 2013
(86) PCT No.: PCT/MY2013/000068

§ 371 Date: Apr. 15, 2015

(30) Foreign Application Priority Data
Oct. 10, 2012 (MY) PI 2012700758

(51) Int. Cl. C12P 7/64; C11B 1/10; C12N 9/90; C12N 15/82; C12N 9/04

(52) U.S. Cl. CPC C12P 7/64; C12N 15/8247; C12N 9/0006; C12N 9/90; C11B 1/10; C12Y 101/01008; C12Y 503/01001

(57) ABSTRACT

Methods are provided for obtaining a genetically modified plant, wherein the plant exhibits an increased oil yield relative to a corresponding control plant that is not so genetically modified. The methods comprise genetically modifying a plant progenitor cell to cause a decrease in triose-phosphate isomerase activity and an increase in glycerol-3-phosphate dehydrogenase activity. The methods also comprise culturing the genetically modified plant progenitor cell to obtain the genetically modified plant. Also provided are methods for increasing oil yield, comprising genetically modifying a plant to cause, in at least one oil-producing organ or tissue of the plant, a decrease in triose-phosphate isomerase activity and an increase in glycerol-3-phosphate dehydrogenase activity. The genetic modification is carried out across more than a single generation. The genetically modified plant exhibits an increased oil yield relative to a corresponding control plant. Also provided are similar methods directed to a microbe.

### TECHNICAL FIELD

This application relates to methods for obtaining a genetically modified plant or microbe and to methods of increasing oil yield, and more particularly to methods for obtaining a genetically modified plant or microbe, wherein the plant or microbe exhibits an increased oil yield relative to a corresponding control plant or microbe that is not so genetically modified, and to methods for increasing oil yield, comprising genetically modifying a plant or microbe.

### BACKGROUND ART

Plants and microbes are important sources of oils. Plants have long been used as sources of oils for foods, industrial products, and fuels. Plant oils include triglycerides, i.e. esters of glycerol and three fatty acids, among other compounds, with chain length and degree of saturation of the fatty acids affecting properties and quality, and thus potential uses. Major oil-producing plants include the African oil palm Elaeis guineensis Jacq., soybean, and rapeseed, which together account for about three-quarters of current world consumption of triglyceride plant oils. Microbes are a promising alternative to plants for large-scale production of certain oils, such as specific polyunsaturated fatty acids, based on shorter generation times, easier genetic manipulation, and greater potential chemical diversity of microbes relative to plants. Major oil producing microbes include the fungus Mortierella alpina, which is used for production of arachidonic acid.

Oil palm is particularly important among oil-food crops. Oil palm plants are monoecious, i.e. single plants produce both male and female flowers, and are characterized by alternating series of male and female inflorescences. The male inflorescence is made up of numerous spikelets, and can bear well over 100,000 flowers. Oil palm is naturally cross-pollinated by insects and wind. The female inflorescence is a spadix which contains several thousands of flowers borne on thorny spikelets. A bunch carries 500 to 4,000 fruits. The oil palm fruit is a sessile drupe that is spherical to ovoid or elongated in shape and is composed of an exocarp, a mesocarp containing palm oil, and an endocarp surrounding a kernel.

Oil palm is important both because of its high yield and because of the high quality of its oil. Regarding yield, oil palm is the highest yielding oil-food crop, with a recent average yield of 3.67 tonnes per hectare per year and with best progenies known to produce about 10 tonnes per hectare per year. Oil palm is also the most efficient plant known for harnessing the energy of sunlight for producing oil. Regarding quality, oil palm is cultivated for both palm oil, which is produced in the mesocarp, and palm kernel oil, which is produced in the kernel. Palm oil in particular is a balanced oil, having almost equal proportions of saturated fatty acids (≈55% including 45% of palmitic acid) and unsaturated fatty acids (≈45%), and it includes beta carotene. The palm kernel oil is more saturated than the mesocarp oil. Both are low in free fatty acids. The current combined output of palm oil and palm kernel oil is about 50 million tonnes per year, and demand is expected to increase substantially in the future with increasing global population and per capita consumption of oils and fats.

Although oil palm is the highest yielding oil-food crop, current oil palm crops produce well below their theoretical maximum. Moreover, conventional methods for identifying potential high-yielding palms for use in crosses to generate progeny with higher yields require cultivation of palms and measurement of production of oil thereby over the course of many years, which is both time and labor intensive. In addition, conventional breeding techniques for propagation of oil palm for oil production are also time and labor intensive, particularly because the most productive, and thus commercially relevant, palms exhibit a hybrid phenotype which makes propagation thereof by direct hybrid crosses impractical. Some of the same problems apply regarding other oil-producing plants too. Oil producing microbes present other problems, including that use of carbon sources derived from plants for cultivation of the microbes tends to be inefficient. Accordingly, a need exists for general approaches to obtain plants and microbes that exhibit increased oil yields.

Genetic modification of plants and microbes may offer solutions. For example, genetic modification of crops such as soy and corn by the introduction of pest resistance genes derived from other organisms is now well known as a means for increasing crop yields generally. Moreover, methods for increasing plant yields by increasing or generating in the plant activities of particular proteins have also been disclosed, for example by Schön et al., WO 2010/046221. In addition, genetic modification of particular oil plants to express particular genes has been reported to result in increased oil yields. For example, Zank et al., US Pub. No. 2009/0083882, discloses methods of increasing the total oil content and/or glycerol 3-phosphate content in transgenic oil crop plants by expressing glycerol-3-phosphate dehydrogenases from yeasts therein and notes that an increase in content of triacylglycerides is achieved by increasing the glycerol-3-phosphate dehydrogenase activity. Also for example, Zou et al., U.S. Pat. No. 6,825,039, discloses a method for increasing oil content in a plant by modulating pyruvate dehydrogenase kinase protein expression, including a step of stably transforming a plant cell with a plant pyruvate dehydrogenase kinase polynucleotide operably linked to a promoter. Also for example, Zou et al., U.S. Pat. No. 7,057,091, discloses a method of changing the oil or biopolymer content of a plant, plant storage organ, or plant seed, the method including introducing a sense or anti-sense nucleic acid construct into a plant transformation vector to produce a modified plant transformation vector, wherein the sense or anti-sense nucleic acid construct includes an isolated, purified, or recombinant nucleic acid encoding a Brassica pyruvate dehydrogenase kinase protein, among other steps. Genetic engineering of microbes for production of diverse compounds is also known. However, given the great diversity of biochemical pathways and regulatory mechanisms across plants and microbes, it is not apparent that approaches for increasing oil yields in specific plant species by genetic modification are generalizable to other plant species, let alone to microbes, or vice versa. It is also not apparent whether particular combinations of approaches, e.g. genetic modification with respect to combinations of genes, may provide for further increases in oil yields, and if so what those combinations may be.

### DISCLOSURE OF INVENTION

in one example embodiment, a method for obtaining a genetically modified plant is disclosed. The plant exhibits an increased oil yield relative to a corresponding control plant that is not so genetically modified. The method comprises genetically modifying a plant progenitor cell to cause a decrease in triose-phosphate isomerase activity and an increase in glycerol-3-phosphate dehydrogenase activity. The method also comprises culturing the genetically modified plant progenitor cell to obtain the genetically modified plant.

In another example embodiment, a method for obtaining a genetically modified microbe is disclosed. The microbe exhibits an increased oil yield relative to a corresponding control microbe that is not so genetically modified. The method comprises genetically modifying a microbial cell to cause a decrease in triose-phosphate isomerase activity and an increase in glycerol-3-phosphate dehydrogenase activity. The method also comprises culturing the microbial cell to obtain the genetically modified microbe.

In another example embodiment, a method for increasing oil yield is disclosed. The method comprises genetically modifying a plant to cause, in at least one oil-producing organ or tissue of the plant, a decrease in triose-phosphate isomerase activity and an increase in glycerol-3-phosphate dehydrogenase activity. The genetic modification is carried out across more than a single generation of the plant. The genetically modified plant exhibits an increased oil yield relative to a corresponding control plant that is not so genetically modified.

In another example embodiment, a method for increasing oil yield is disclosed. The method comprises genetically modifying a microbe to cause a decrease in triose-phosphate isomerase activity and an increase in glycerol-3-phosphate dehydrogenase activity in the microbe. The genetic modification is carried out across more than a single generation of the microbe. The genetically modified microbe exhibits an increased oil yield relative to a corresponding control microbe that is not so genetically modified.

### BRIEF DESCRIPTION OF DRAWINGS

FIG. 1 is a graph of relative amounts of triose-phosphate isomerase protein (density) versus time (12 to 22 weeks after pollination), for mesocarp tissue of fruits of high-yielding oil palm plants (diamonds) versus low-yielding oil palm plants (X symbols). Statistically significant differences are noted (stars).

FIG. 2 is a graph of relative amounts of fructose-1,6-bisphosphate aldolase protein (density) versus time (12 to 22 weeks after pollination), for mesocarp tissue of fruits of high-yielding oil palm plants (diamonds) versus low-yielding oil palm plants (X symbols). Statistically significant differences are noted (stars).

FIG. 3 is a graph of relative amounts of ATP citrate lyase protein (expression intensity) versus time (12 to 22 weeks after pollination), for mesocarp tissue of fruits of high-yielding oil palm plants (squares) versus low-yielding oil palm plants (X symbols). Statistically significant differences are noted (stars).

FIG. 4 is a graph of relative amounts of pyruvate kinase protein (expression intensity) versus time (12 to 22 weeks after pollination), for mesocarp tissue of fruits of high-yielding oil palm plants (squares) versus low-yielding oil palm plants (X symbols). Statistically significant differences are noted (stars).

FIG. 5 is a graph of relative amounts of aconitase protein (expression intensity) versus time (12 to 22 weeks after pollination), for mesocarp tissue of fruits of high-yielding oil palm plants (squares) versus low-yielding oil palm plants (X symbols). Statistically significant differences are noted (stars).

FIG. 6 is a graph of total lipid content (grams per gram of biomass) for yeast strains corresponding to wild-type (1), a strain that over-expresses fructose-1,6-bisphosphate aldolase (2), a strain that over-expresses glycerol-3-phosphate dehydrogenase (3), a strain that over-expresses cytosolic triose-phosphate isomerase (4), a strain that over-expresses plastidial triose-phosphate isomerase (5), and a strain that over-expresses glyceraldehyde-3-phosphate dehydrogenase (6). Error bars are shown.

FIG. 7 is a graph of relative concentration of 16:0 fatty acids, i.e. saturated 16-carbon fatty acids (expressed as relative abundance), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 8 is a graph of relative concentration of 18:0 fatty acids, i.e. saturated 18-carbon fatty acids (expressed as relative abundance), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 9 is a graph of relative concentration of 18:1 fatty acids, i.e. mono-unsaturated 18-carbon fatty acids (expressed as relative abundance), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 10 is a graph of concentration of fructose 1,6-bisphosphate (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 11 is a graph of concentration of glycerol 3-phosphate (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 12 is a graph of concentration of 3-phosphoglyceric acid (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 13 is a graph of concentration of malic acid (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 14 is a graph of concentration of isocitric acid (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 15 is a graph of concentration of 2-oxoglutaric acid (expressed as nmol/g dry weight), versus time (12 to 22 weeks after pollination), for mesocarp tissue of fruits of high-yielding oil palm plants (triangles) versus low-yielding oil palm plants (circles). Error bars are shown. Statistically significant differences based on a p-value<0.005 are noted (*).

FIG. 16 is a graph of the ratios of malic acid to citric acid versus time (12 to 22 weeks after pollination), for mesocarp tissue of fruits of high-yielding oil palm plants (triangles) versus low-yielding oil palm plants (circles) (n=8).

### BEST MODE FOR CARRYING OUT THE INVENTION

The application is drawn to methods for obtaining a genetically modified plant or microbe, wherein the plant or microbe exhibits an increased oil yield relative to a corresponding control plant or microbe that is not so genetically modified, comprising genetically modifying a plant progenitor cell or a microbial cell to cause a decrease in triose-phosphate isomerase activity and an increase in glycerol-3-phosphate dehydrogenase activity. This application is also drawn to methods for increasing oil yield, comprising genetically modifying a plant or microbe to cause, in at least one oil-producing organ or tissue of the plant, or in the microbe, a decrease in triose-phosphate isomerase activity and an increase in glycerol-3-phosphate dehydrogenase activity.

By studying and comparing gene expression, protein levels, and metabolite levels in populations of oil palm that yield high amounts of oil (“high-yielding”) versus those that yield low amounts of oil (“low-yielding”), in combination with manipulation of gene expression in a yeast model system, it has been determined that decreasing triose-phosphate isomerase activity in combination with increasing glycerol-3-phosphate dehydrogenase activity, e.g. by genetic modification, can lead to increased oil production in plants and microbes in general. Without wishing to be bound by theory, it is believed that this occurs by increasing carbon flux toward dihydroxyacetone phosphate at a critical branch point in biosynthesis, based on the decrease in triose-phosphate isomerase activity, combined with converting dihydroxyacetone phosphate to glycerol 3-phosphate efficiently, based on the increase in glycerol-3-phosphate dehydrogenase activity. It is believed that this combination in particular allows for increased production of glycerol 3-phosphate, which provides the backbone molecule of lipids including triglycerides, without having dihydroxyacetone phosphate accumulate to toxically high levels.

It has also been determined that increasing malate dehydrogenase activity and/or increasing ATP citrate lyase activity, e.g. again by genetic modification, can lead to further increased oil production in plants and microbes in general. Without wishing to be bound by theory, it is believed that increasing malate dehydrogenase activity increases conversion of malic acid to citric acid, and that increasing ATP citrate lyase activity provides for increased conversion of citric acid to acetyl-CoA for fatty acid biosynthesis, with each also contributing to increased oil yield in plants and microbes in general.

It has also been determined that decreasing glyceraldehyde-3-phosphate dehydrogenase activity, increasing fructose-1,6-bisphosphate aldolase activity, increasing pyruvate kinase activity, and/or increasing aconitase activity, e.g. again by genetic modification, can lead to further increased oil production in plants and microbes in general. Without wishing to be bound by theory, it is believed that decreasing glyceraldehyde-3-phosphate dehydrogenase activity causes a further shift in the equilibrium toward dihydroxyacetone phosphate by slowing consumption of glyceraldehyde-3-phosphate, that increasing fructose-1,6-bisphosphate aldolase activity causes increased flux through the glycolytic pathway, that increasing pyruvate kinase activity causes increased production of pyruvate for carbon supply to the TCA cycle and acetyl-CoA, and that increasing aconitase activity may benefit equilibria in the TCA cycle, with each further contributing to increased oil yield in plants and microbes in general.

Because the studies and comparisons of gene expression, protein levels, and metabolite levels were carried out in populations of oil palm in particular, and because oil palm is important among oil-food crops both because of its high yield and because of the high quality of its oil, it is believed that the methods for obtaining a genetically modified plant and the methods for increasing oil yield, as disclosed herein, are useful, for example, for increasing oil yields of oil crop plants generally. Moreover, because the studies and comparisons were carried out in combination with manipulation of gene expression in a yeast model system, it is believed that the methods for obtaining a genetically modified microbe and the methods for increasing oil yield, as disclosed herein, are useful, for example, for increasing oil yields of oleaginous microbes generally.

The term “oil” as used herein includes lipids, fats, fatty acid mixtures, and triglycerides, among other oil compounds. An oil can include saturated fatty acids and/or unsaturated fatty acids, e.g. in esterified forms as components of triglycerides. The composition of an oil, including the profile of fatty acids thereof, can reflect the plant or microbe from which it is derived.

The term “oleaginous microbe” refers to a microbe that can accumulate more than 20% of its biomass as oil (e.g. >30%, >40%, >50%, >60%, or >70%).

The singular forms “a,” “an,” and “the” are intended to include the plural forms as well, unless the context clearly indicates otherwise.

As noted above, a method is provided for obtaining a genetically modified plant, wherein the plant exhibits an increased oil yield relative to a corresponding control plant that is not so genetically modified. The genetically modified plant so obtained can be, for example, an oil crop, such as oil palm, olive, coconut palm, soybean, sunflower, rapeseed, field mustard, canola, peanut, corn, cotton, safflower, castor, jatropha, or camelina. The plant can also be, for example, a non-oil crop. As will be appreciated, the plant can be an individual plant and/or a plurality of plants.

The control plant can be a plant that has not been genetically modified in the same way as the plant that exhibits the increased oil yield but that is otherwise genetically similar or identical thereto. For example, the control plant can be of the same species, cultivar, variety, group of progeny, and/or clone as the genetically modified plant, though lacking the particular genetic modification of the plant that exhibits an increased oil yield. Also for example, the control plant can also be the product of genetic modification, e.g. including one or more genetic modifications relative to a non-genetically modified plant, though again lacking the particular genetic modification of the plant that exhibits an increased oil yield.

In accordance with the method, the oil yield of the genetically modified plant can be increased by at least 10% relative to the corresponding control plant. For example, the oil yield of the genetically modified plant can be increased by at least 20%, 30%, 40%, 50%, or even more, relative to the corresponding control plant. The oil yield can also be increased without causing a deleterious shift in the profile of fatty acids of the oil, e.g. without causing a deleterious shift in the profile of C16:0, C18:0, and/or C18:1 fatty acids.

The method can comprise genetically modifying a plant progenitor cell to cause a decrease in triose-phosphate isomerase activity and an increase in glycerol-3-phosphate dehydrogenase activity. By genetically modifying the plant progenitor cell, it is meant that the genetic material of the plant progenitor cell is modified or varied, at least in part, by direct manipulation, e.g. by addition, deletion, or other modification of DNA sequences, using techniques of molecular biology, such as transformation, mutagenesis, and/or recombination, in order to introduce variation. This is in contrast to relying only on non-genetic modification techniques, such as classical breeding techniques and the like, to introduce variation.

Triose-phosphate isomerase catalyzes the interconversion of glyceraldehyde 3-phosphate and dihydroxyacetone phosphate in plant cell cytosol and plastids and plays an important role in metabolism by replenishing glyceraldehyde-3-phosphate in plant cells as it is consumed during glycolysis. Accordingly, the decrease in triose-phosphate isomerase activity can result in a relative increase in dihydroxyacetone phosphate in plant cells. In some embodiments, the triose-phosphate isomerase activity can be based, for example, on a cytosolic isoform of triose-phosphate isomerase or a plastidial isoform of triose-phosphate isomerase.

Glycerol-3-phosphate dehydrogenase catalyzes the reversible redox conversion of dihydroxyacetone phosphate to glycerol 3-phosphate and serves to link carbohydrate metabolism and lipid metabolism. Accordingly, the increase in glycerol-3-phosphate dehydrogenase activity can result in an increase in glycerol 3-phosphate available for lipid production. In some embodiments, the glycerol-3-phosphate dehydrogenase activity can be based, for example, on a plastidial isoform of glycerol-3-phosphate dehydrogenase.

The plant progenitor cell can be a plant cell that is subject to genetic modification and from which the plant that exhibits an increased oil yield can be derived. The plant progenitor cell can be, for example, an isolated cell, a cell of a plant leaf, a cell of a plant flower, or a cell of a plant embryo. Also for example, the plant progenitor cell can be a cell of a plant seed, a cell of a young plant, a cell within a tissue that has been isolated from a plant, and/or a cell that itself has been isolated from a plant.

The plant progenitor cell can be genetically modified by one or more of various methods, e.g. as discussed below, to yield the plant that exhibits an increased oil yield. The genetic modification may be carried out on more than one plant progenitor cell.

The method can also comprise culturing the genetically modified plant progenitor cell to obtain the genetically modified plant. The culturing can be carried out, for example, by methods of plant tissue culture and plant regeneration. For example, the genetically modified plant progenitor cell can be placed on a selectable rooting and shooting medium or the like, under conditions suitable for regeneration of a plant.

In accordance with the method, the genetic modification can further cause at least one of an increase in malate dehydrogenase activity and an increase in ATP citrate lyase activity. Malate dehydrogenase oxidizes malate to oxaloacetate and produces NADH as the final step of the TCA cycle. Accordingly, the increase in malate dehydrogenase activity can result in increased conversion of malic acid to citric acid. ATP citrate lyase catalyzes conversion of citrate, ATP, CoA, and water to oxaloacetate, acetyl-CoA, ADP, and orthophosphate, linking carbohydrate metabolism and fatty acid metabolism. Accordingly, the increase in ATP citrate lyase activity can result in increased conversion of citric acid to acetyl-CoA for fatty acid biosynthesis. In some examples, the genetic modification can cause both the increase in malate dehydrogenase activity and the increase in ATP citrate lyase activity.

In accordance with the method, the genetic modification can further cause at least one of a decrease in glyceraldehyde-3-phosphate dehydrogenase activity, an increase in fructose-1,6-bisphosphate aldolase activity, an increase in pyruvate kinase activity, and an increase in aconitase activity. Glyceraldehyde-3-phosphate dehydrogenase catalyzes the conversion of glyceraldehyde 3-phosphate to 1,3-bisphosphoglycerate, linked directly to reduction of NAD⁺ to NADH, in glycolysis. Accordingly, the decrease in glyceraldehyde-3-phosphate dehydrogenase activity can further enhance an equilibrium shift toward production of dihydroxyacetone phosphate and subsequently increase the production of the glycerol 3-phosphate backbone molecule of lipids. Fructose-1,6-bisphosphate aldolase catalyzes the aldol cleavage of fructose 1,6-bisphosphate to glyceraldehyde 3-phosphate and dihydroxyacetone phosphate. Accordingly, the increase in fructose-1,6-bisphosphate aldolase activity can increase carbon flux through the glycolytic pathway to increase conversion of sugars to lipids. Pyruvate kinase catalyzes the transfer of the phosphate group from phosphoenolpyruvate to ADP to form ATP and pyruvate in glycolysis. Accordingly, the increase in pyruvate kinase activity can result in increased production of pyruvate to supply carbon to the TCA cycle and for acetyl-CoA. Aconitase catalyzes stereo-specific isomerization of citrate to isocitrate in the TCA cycle. Accordingly, the increase in aconitase activity may benefit equilibria in the TCA cycle. In some examples, the genetic modification can cause at least two or at least three of the decrease in glyceraldehyde-3-phosphate dehydrogenase activity, the increase in fructose-1,6-bisphosphate aldolase activity, the increase in pyruvate kinase activity, and the increase in aconitase activity. Also in some examples, the genetic modification can cause all four of the decrease in glyceraldehyde-3-phosphate dehydrogenase activity, the increase in fructose-1,6-bisphosphate aldolase activity, the increase in pyruvate kinase activity, and the increase in aconitase activity.

In accordance with the method, at least one of the decreases in activity and at least one of the increases in activity can contribute to confer the increased oil yield of the genetically modified plant. The decrease in activity can be any one or more of the decreases noted above, i.e. the decrease in triose-phosphate isomerase activity and the decrease in glyceraldehyde-3-phosphate dehydrogenase activity. Similarly, the increase in activity can be any one or more of the increases noted above, i.e. the increase in glycerol-3-phosphate dehydrogenase activity, the increase in malate dehydrogenase activity, the increase in ATP citrate lyase activity, the increase in fructose-1,6-bisphosphate aldolase activity, the increase in pyruvate kinase activity, and the increase in aconitase activity. By the decrease in activity and increase in activity contributing to confer the increased oil yield of the genetically modified plant, it is meant that both the decrease and the increase contribute, i.e. that neither the decrease alone nor the increase alone accounts for the entire increased oil yield of the plant. Thus, in some examples, the decrease in triose-phosphate isomerase activity and the increase in glycerol-3-phosphate dehydrogenase activity together can contribute to confer the increased oil yield of the genetically modified plant. Alternatively or additionally, in some examples the decrease in glyceraldehyde-3-phosphate dehydrogenase activity and the increase in fructose-1,6-bisphosphate aldolase activity can contribute to confer the increased oil yield.

In accordance with the method, all of the decreases in activity and the increases in activity can occur in at least one oil-producing organ or tissue of the genetically modified plant. The oil-producing organ or tissue can be an organ or tissue that produces and/or stores substantial amounts of oil during part or all of the life of a corresponding plant. The oil-producing organ or tissue can be, for example, fruit, mesocarp, kernel, or seed. Thus, for example, in oil palm plants the mesocarp tissue and the kernel tissue are exemplary oil-producing tissues. As will be appreciated, the above-noted decreases in activity and increases in activity may be expected to have a substantial effect on oil production in plants to the extent that the decreases and increases occur in an organ or tissue that produces oil. This may allow the decreases and increases to most directly affect oil production and thus oil yields.

In accordance with the method, all of the decreases in activity and the increases in activity can occur in at least one oil-producing organ or tissue of the genetically modified plant at least during onset of oil deposition in the oil-producing organ or tissue. The timing of onset of oil deposition in the oil-producing organ or tissue can be determined, for example, based on measuring concentrations of oil in an organ or tissue directly, measuring enzymatic activities and/or metabolite levels indicative of the onset of oil deposition in an organ or tissue, and/or based on predicting the onset of oil deposition relative to a developmental stage or other biological stage of an organ or tissue, e.g. time after pollination of a flower of a plant.

Thus, for example, in oil palm onset of oil deposition follows pollination according to the following time frame. Oil deposition in the endosperm starts at approximately 12 weeks after pollination and is almost complete by 16 weeks after pollination. Oil deposition in mesocarp starts at approximately 15 weeks after pollination and continues until fruit maturity at about 20 weeks after pollination. More specifically, 12 weeks after pollination marks the start of oil deposition in endosperm but precedes the start of oil deposition in mesocarp, 16 weeks after pollination marks the point of highest transcript expression level in mesocarp, following the initiation of oil biosynthesis after pollination, and 18 weeks after pollination marks the time at which transcript expression would be expected to decrease as the fruit matures. As will be appreciated, onset of oil deposition can proceed differently, e.g. in different tissues and/or according to a different time frame, in organs and tissues of other plants.

The method can further comprise selecting the genetically modified plant based on at least one of the decreases in activity and the increases activity. By this it is meant that the selecting can be based on any one or more of the decrease in triose-phosphate isomerase activity, the decrease in glyceraldehyde-3-phosphate dehydrogenase activity, the increase in glycerol-3-phosphate dehydrogenase activity, the increase in malate dehydrogenase activity, the increase in ATP citrate lyase activity, the increase in fructose-1,6-bisphosphate aldolase activity, the increase in pyruvate kinase activity, and the increase in aconitase activity. The selecting can be carried out, for example, by measuring the one or more decreases in activity or increases in activity in the organ or tissue of the genetically modified plant relative to the corresponding organ or tissue of the control plant, in order to determine whether the genetically modified plant exhibits the one or more decreases or increases, and if so then propagating the genetically modified plant, and if not then not propagating the genetically modified plant. As will be appreciated, such measurements can be carried out by various methods, for example by measuring levels of expression of genes encoding the corresponding activities, e.g. by reverse transcription polymerase chain reaction (also termed RT-PCR), RNA-seq, hybridization, or microarray, by measuring levels of proteins corresponding to the activities, e.g. by two-dimensional fluorescence difference gel electrophoresis and/or antibody-based detection, or by measuring the activities directly, e.g. by enzymatic assay. Moreover, the measurements and/or selection can be applied to individual plants or multiple plants.

In accordance with the method, at least one of the decreases in activity can be based, for example, on a technique such as mutagenesis, RNAi, expression of siRNA, gene silencing, homologous recombination, disruption of a regulatory sequence, partial gene deletion, or full gene deletion. Mutagenesis can include adding, deleting, and or changing one or more nucleotides in one or more genes of a plant to alter the one or more genes. RNAi can include introducing one or more RNAs, such as an siRNA, into a plant cell to decrease the expression of one or more genes of a plant. Expression of siRNA can include expressing one or more siRNAs in a plant cell to decrease the expression of one or more genes of a plant. Gene silencing can include expressing one or more RNA sequences within a plant cell to decrease the expression of one or more genes of a plant. Homologous recombination can include exchanging one or more modified nucleotide sequences for one or more naturally occurring nucleotide sequences to alter one or more genes in a plant. Disruption of a regulatory sequence can include adding, deleting, and or changing one or more nucleotides of a regulatory sequence such as a promoter or enhancer to alter expression of one or more genes in a plant. Partial or full gene deletion can include deleting part or all, respectively, of one or more genes of a plant. As will be appreciated, each of these techniques can be used to cause at least one of the decreases in activity. For example, one or more of these techniques can be carried out alone or in combination in the progenitor plant cell to genetically modify the cell, such that the plant derived therefrom exhibits at least one of the decreases in activity.

In accordance with the method, at least one of the increases in activity can be based, for example, on a technique such as transformation, Agrobacterium-mediated transformation, viral transformation, particle bombardment, introduction of recombinant DNA, introduction of a plasmid, or introduction of an artificial chromosome. Transformation can include introducing one or more nucleotide sequences, e.g. a gene, into a cell, e.g. by Agrobacterium-mediated transformation, viral transformation, particle bombardment, or other suitable transformation method, wherein the one or more nucleotide sequences are stably maintained following introduction and wherein subsequent expression of the one or more nucleotide sequences results in at least one of the increases in activity. Introduced nucleotide sequences can be, for example, recombinant DNA, a plasmid, or an artificial chromosome, among others. As will be appreciated, each of these techniques can be used to cause at least one of the increases in activity. For example, one or more of these techniques can be carried out alone or in combination in the progenitor plant cell to genetically modify the cell, such that the plant derived therefrom exhibits at least one of the increases in activity.

In accordance with the method, at least one of the decreases in activity can be based, for example, on an effect selected from the group consisting of a decrease in specific activity of a corresponding enzyme, a decrease in copy number of a corresponding gene, a deleterious mutation in a corresponding gene, a deleterious modification of a corresponding enzyme, or a decrease in transcription of a corresponding gene. Thus, for example, a particular enzymatic activity, e.g. triose-phosphate isomerase activity, can be decreased based on a decrease in the specific activity, i.e. activity/mass, of the corresponding enzyme, e.g. triose-phosphate isomerase enzyme, for example based on a mutation that decreases the catalytic efficiency of the enzyme. Also for example, the particular enzymatic activity can be decreased based on a decrease in copy number of a corresponding gene, e.g. triose-phosphate isomerase gene, for example based on deletion of one or more copies of the gene. Also for example, the particular enzymatic activity can be decreased based on a deleterious mutation in a corresponding gene, for example based on a mutation that results in amino acid substitution that decreases the specific activity of the corresponding enzyme. Also for example, the particular enzymatic activity can be decreased based on a deleterious modification of the corresponding enzyme, based for example on a post-translational modification that decreases the specific activity of the enzyme. Also for example, the particular enzymatic activity can be decreased based on a decrease in transcription of the corresponding gene, for example based on a mutation that decreases the efficiency of a regulatory sequence, e.g. a promoter or enhancer, of the gene.

In accordance with the method, at least one of the increases in activity can be based, for example, on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene, conversely as discussed above regarding decreases in activity.

Also disclosed is a method of producing oil from a genetically modified plant that exhibits an increased oil yield relative to a corresponding control plant that is not so genetically modified. The method can comprise obtaining the genetically modified plant by the method for obtaining a genetically modified plant, as described above. The method can also comprise extracting oil from an oil-producing organ or tissue of the genetically modified plant. The step of extracting oil can be carried out, for example, following harvesting of a plant that has been cultured or grown in the field and/or by approaches that are known in the art, e.g. for oil palm plants based on harvesting of fruit bunches followed by extraction of oil, within 24 hours, from fresh and non-wounded fruits thereof.

Also disclosed is a genetically modified plant that exhibits an increased oil yield relative to a corresponding control plant that is not so genetically modified. The genetically modified plant can be obtained according to the method for obtaining a genetically modified plant, as described above. The oil yield of the genetically modified plant can be increased by at least 10% relative to the corresponding control plant. For example, the oil yield of the genetically modified plant can be increased by at least 20%, 30%, 40%, 50%, or even more, relative to the corresponding control plant.

As also noted above, a method is also provided for obtaining a genetically modified microbe, wherein the microbe exhibits an increased oil yield relative to a corresponding control microbe that is not so genetically modified. The microbe that is genetically modified can be, for example, an oleaginous microbe, an oleaginous bacterium, an oleaginous actinomycetes, an oleaginous Mycobacterium, an oleaginous Streptomyces, an oleaginous Rhodococcus, an oleaginous Nocardia, an oleaginous fungus, an oleaginous yeast, or an oleaginous Mortierella. As will be appreciated, the microbe can be an individual microbe and/or a plurality of microbes.

Similarly as discussed above for plants, the microbe exhibits an increased oil yield relative to a corresponding control microbe that is not so genetically modified. The control microbe can be a microbe that has not been genetically modified in the same way as the microbe that exhibits the increased oil yield but that is otherwise genetically similar or identical thereto. For example, the control microbe can be of the same species, strain, group of progeny, and/or clone as the genetically modified microbe, though lacking the particular genetic modification of the microbe that exhibits an increased oil yield. Also for example, the control microbe can also be the product of genetic modification, e.g. including one or more genetic modifications relative to a non-genetically modified microbe, though again lacking the particular genetic modification of the microbe that exhibits an increased oil yield.

In accordance with the method, the oil yield of the genetically modified microbe can be increased by at least 10% relative to the corresponding control microbe. For example, the oil yield of the genetically modified microbe can be increased by at least 20%, 30%, 40%, 50%, or even more, relative to the corresponding control microbe.

Similarly as discussed above regarding plants, the method can comprise genetically modifying a microbial cell to cause a decrease in triose-phosphate isomerase activity and an increase in glycerol-3-phosphate dehydrogenase activity. By genetically modifying the microbe, it is meant that the genetic material of the microbe is modified or varied by direct manipulation, e.g. by addition, deletion, or other modification of DNA sequences, using techniques of molecular biology, such as transformation, mutagenesis, and/or recombination, to introduce variation. This is in contrast to relying only on non-genetic modification techniques, such as classical microbial selection techniques and the like, to introduce variation.

The microbial cell can be a cell that is subject to genetic modification and from which the microbe that exhibits an increased oil yield can be derived. The microbial cell can be, for example, an isolated cell, a cell in a culture, or a cell in a colony. The microbial cell can be genetically modified by one or more of various methods to yield the microbe that exhibits an increased oil yield. Like for plant progenitor cells, the genetic modification may be carried out on more than microbial cell.

The method can also comprise culturing the genetically modified microbial cell to obtain the genetically modified microbe. The culturing can be carried out by methods of microbial cultivation. For example, the genetically modified microbial cell can be added to a liquid cultivation medium, under conditions suitable for survival, growth, and reproduction of a microbe.

In accordance with the method, the genetic modification can further cause at least one of an increase in malate dehydrogenase activity and an increase in ATP citrate lyase activity, as discussed above. Thus, in some examples, the genetic modification can cause both the increase in malate dehydrogenase activity and the increase in ATP citrate lyase activity.

In accordance with the method, the genetic modification can further cause at least one of a decrease in glyceraldehyde-3-phosphate dehydrogenase activity, an increase in fructose-1,6-bisphosphate aldolase activity, an increase in pyruvate kinase activity, and an increase in aconitase activity, as discussed above. Thus, in some examples, the genetic modification can cause at least two or at least three of the decrease in glyceraldehyde-3-phosphate dehydrogenase activity, the increase in fructose-1,6-bisphosphate aldolase activity, the increase in pyruvate kinase activity, and the increase in aconitase activity. Also in some examples, the genetic modification can cause all four of the decrease in glyceraldehyde-3-phosphate dehydrogenase activity, the increase in fructose-1,6-bisphosphate aldolase activity, the increase in pyruvate kinase activity, and the increase in aconitase activity.

In accordance with the method, at least one of the decreases in activity and at least one of the increases in activity can contribute to confer the increased oil yield of the genetically modified microbe, similarly as discussed above. Thus, the decrease in activity can be any one or more of the decreases noted above, i.e. the decrease in triose-phosphate isomerase activity and the decrease in glyceraldehyde-3-phosphate dehydrogenase activity. Likewise, the increase in activity can be any one or more of the increases noted above, i.e. the increase in glycerol-3-phosphate dehydrogenase activity, the increase in malate dehydrogenase activity, the increase in ATP citrate lyase activity, the increase in fructose-1,6-bisphosphate aldolase activity, the increase in pyruvate kinase activity, and the increase in aconitase activity. Also, by the decrease in activity and increase in activity contributing to confer the increased oil yield of the genetically modified microbe, it is meant that both the decrease and the increase contribute, i.e. that neither the decrease alone nor the increase alone accounts for the entire increased oil yield of the microbe. Thus, in some examples, the decrease in triose-phosphate isomerase activity and the increase in glycerol-3-phosphate dehydrogenase activity together can contribute to confer the increased oil yield of the genetically modified microbe. Alternatively or additionally, in some examples the decrease in glyceraldehyde-3-phosphate dehydrogenase activity and the increase in fructose-1,6-bisphosphate aldolase activity can contribute to confer the increased oil yield.

The method can further comprise selecting the genetically modified microbe based on at least one of the decreases in activity and the increases in activity, as discussed above.

In accordance with the method, at least one of the decreases in activity can be based on a technique selected from the group consisting of mutagenesis, RNAi, expression of siRNA, gene silencing, homologous recombination, disruption of a regulatory sequence, partial gene deletion, and full gene deletion, as discussed above.

In accordance with the method, at least one of the increases in activity can be based on a technique selected from the group consisting of transformation, electroporation, transduction, introduction of recombinant DNA, introduction of a plasmid, and introduction of an artificial chromosome. Transformation can include introducing one or more nucleotide sequences, e.g. a gene, into a cell, e.g. by electroporation, transduction, or other suitable transformation method, wherein the one or more nucleotide sequences are stably maintained following introduction and wherein subsequent expression of the one or more nucleotide sequences results in at least one of the increases in activity. Again, introduced nucleotide sequences can be, for example, recombinant DNA, a plasmid, or an artificial chromosome, among others. As discussed above, each of these techniques can be used, alone or in combination, to cause at least one of the increases in activity.

In accordance with the method, at least one of the decreases in activity can be based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene, as discussed above. Also, at least one of the increases in activity can be based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene, as discussed above.

Also disclosed is a method of producing oil from a genetically modified microbe that exhibits an increased oil yield relative to a corresponding control microbe that is not so genetically modified. The method can comprise obtaining the genetically modified microbe by the method for obtaining a genetically modified microbe as described above. The method can also comprise extracting oil from the genetically modified microbe. The step of extracting oil may be carried out by approaches that are known in the art, e.g. based on chloroform extraction of the oil from the microbe.

Also disclosed is a genetically modified microbe that exhibits an increased oil yield relative to a corresponding control microbe that is not so genetically modified. The genetically modified microbe can be obtained according to the method for genetically modifying a microbe as described above. The oil yield of the genetically modified microbe can be increased by at least 10% relative to the corresponding control microbe. For example, the oil yield of the genetically modified microbe can be increased by at least 20%, 30%, 40%, 50%, or even more, relative to the corresponding control microbe.

As also noted above, a method is also disclosed for increasing oil yield, comprising genetically modifying a plant to cause, in at least one oil-producing organ or tissue of the plant, a decrease in triose-phosphate isomerase activity and an increase in glycerol-3-phosphate dehydrogenase activity. In some embodiments, the triose-phosphate isomerase activity can be based, for example, on a cytosolic isoform of triose-phosphate isomerase or a plastidial isoform of triose-phosphate isomerase. In some embodiments, the glycerol-3-phosphate dehydrogenase activity can be based, for example, on a plastidial isoform of glycerol-3-phosphate dehydrogenase.

The plant that is genetically modified can be, for example, an oil crop, such as oil palm, olive, coconut palm, soybean, sunflower, rapeseed, field mustard, canola, peanut, corn, cotton, safflower, castor, jatropha, or camelina. The plant can also be, for example, a non-oil crop. The plant can be an individual plant and/or a plurality of plants.

In accordance with the method, the genetic modification of the plant can be carried out across more than a single generation of the plant. For example, the genetic modification can be carried out in two or more steps, with a step of propagation of a progenitor of the genetically modified plant carried out therebetween, as follows. A first step of genetic modification can be carried out on one or more cells of a first progenitor of the plant. Then the genetically modified cells of the first progenitor can be propagated to yield a second progenitor of the plant. Then a second step of genetic modification can be carried out on one or more cells of the second progenitor of the plant. Then the genetically modified cells of the second progenitor can be propagated to yield the genetically modified plant. As will be appreciated, any particular step of genetic modification can include introduction of one or more genetic modifications, e.g. modifications of one or more genes. Propagation can be carried out by methods known in the art. Such methods include, for example, asexual propagation, such as vegetative propagation or apomixis. Such methods also include, for example, sexual propagation, such as crossing.

In accordance with the method, the genetically modified plant can exhibit an increased oil yield relative to a corresponding control plant that is not so genetically modified. For example, the oil yield of the genetically modified plant can be increased by at least 10% relative to the corresponding control plant. For example, the oil yield of the genetically modified plant can be increased by at least 20%, 30%, 40%, 50%, or even more, relative to the corresponding control plant. The oil yield can also be increased without causing a deleterious shift in the profile of fatty acids of the oil, e.g. without causing a deleterious shift in the profile of C16:0, C18:0, and/or C18:1 fatty acids.

In accordance with the method, the genetic modification can further cause at least one of an increase in malate dehydrogenase activity and an increase in ATP citrate lyase activity, as discussed above. Thus, in some examples the genetic modification can cause both the increase in malate dehydrogenase activity and the increase in ATP citrate lyase activity.

In accordance with the method, the genetic modification can further cause at least one of a decrease in glyceraldehyde-3-phosphate dehydrogenase activity, an increase in fructose-1,6-bisphosphate aldolase activity, an increase in pyruvate kinase activity, and an increase in aconitase activity. Thus, in some examples the genetic modification can cause at least two or at least three of the decrease in glyceraldehyde-3-phosphate dehydrogenase activity, the increase in fructose-1,6-bisphosphate aldolase activity, the increase in pyruvate kinase activity, and the increase in aconitase activity. Also in some examples, the genetic modification can cause all four of the decrease in glyceraldehyde-3-phosphate dehydrogenase activity, the increase in fructose-1,6-bisphosphate aldolase activity, the increase in pyruvate kinase activity, and the increase in aconitase activity.

In accordance with the method, at least one of the decreases in activity and at least one of the increases in activity can contribute to confer the increased oil yield of the genetically modified plant. Thus, in some examples, the decrease in triose-phosphate isomerase activity and the increase in glycerol-3-phosphate dehydrogenase activity together can contribute to confer the increased oil yield of the genetically modified plant. Alternatively or additionally, in some examples the decrease in glyceraldehyde-3-phosphate dehydrogenase activity and the increase in fructose-1,6-bisphosphate aldolase activity can contribute to confer the increased oil yield.

In accordance with the method, all of the decreases in activity and the increases in activity can occur in the at least one oil-producing organ or tissue of the genetically modified plant, as discussed above. The oil-producing organ or tissue can be, for example, fruit, mesocarp, kernel, or seed. Also, all of the decreases in activity and the increases in activity can occur in the at least one oil-producing organ or tissue of the genetically modified plant at least during onset of oil deposition in the oil-producing organ or tissue.

The method can further comprise selecting the genetically modified plant based on at least one of the decreases in activity and the increases activity, as discussed above. At least one of the decreases in activity can be based on a technique such as mutagenesis, RNAi, expression of siRNA, gene silencing, homologous recombination, disruption of a regulatory sequence, partial gene deletion, or full gene deletion. Also, at least one of the increases in activity can be based on a technique such as transformation, Agrobacterium-mediated transformation, viral transformation, particle bombardment, introduction of recombinant DNA, introduction of a plasmid, or introduction of an artificial chromosome. In addition, at least one of the decreases in activity can be based on an effect such as a decrease in specific activity of a corresponding enzyme, a decrease in copy number of a corresponding gene, a deleterious mutation in a corresponding gene, a deleterious modification of a corresponding enzyme, or a decrease in transcription of a corresponding gene. Furthermore, at least one of the increases in activity can be based on an effect such as an increase in specific activity of a corresponding enzyme, an increase in copy number of a corresponding gene, an advantageous mutation in a corresponding gene, an advantageous modification of a corresponding enzyme, and an increase in transcription of a corresponding gene.

Also disclosed is a method of producing oil. The method can comprise obtaining a genetically modified plant by the method for increasing oil yield, as described above. The method can also comprise extracting oil from the oil-producing organ or tissue of the genetically modified plant, e.g. by methods known in the art.

Also disclosed is a genetically modified plant that exhibits an increased oil yield relative to a corresponding control plant that is not so genetically modified. The genetically modified plant can be obtained according to the method for increasing oil yield, as described above. The oil yield of the genetically modified plant can be increased by at least 10% relative to the corresponding control plant. For example, the oil yield of the genetically modified plant can be increased by at least 20%, 30%, 40%, 50%, or even more, relative to the corresponding control plant.

As also noted, a method is also disclosed for increasing oil yield, comprising genetically modifying a microbe to cause a decrease in triose-phosphate isomerase activity and an increase in glycerol-3-phosphate dehydrogenase activity in the microbe. The microbe that is genetically modified can be, for example, an oleaginous microbe, an oleaginous bacterium, an oleaginous actinomycetes, an oleaginous Mycobacterium, an oleaginous Streptomyces, an oleaginous Rhodococcus, an oleaginous Nocardia, an oleaginous fungus, an oleaginous yeast, or an oleaginous Mortierella. The microbe can be an individual microbe and/or a plurality of microbes.

In accordance with the method, the genetic modification can be carried out across more than a single generation of the microbe. For example, similarly to plants, the genetic modification can be carried out in two or more steps, with a step of propagation of a progenitor of the genetically modified microbe carried out therebetween, as follows. A first step of genetic modification can be carried out on one or more microbial cells. Then the genetically modified cells can be propagated. Then a second step of genetic modification can be carried out on one or more of the propagated cells, to yield the genetically modified microbe. As will be appreciated, any particular step of genetic modification can include introduction of one or more genetic modifications, e.g. modifications of one or more genes. Propagation can be carried out by methods known in the art. Such methods include, for example, asexual propagation, such as by cultivation in a liquid culture medium or on solid culture medium. Such methods also include, for example, sexual propagation, such as crossing.

In accordance with the method, the genetically modified microbe can exhibit an increased oil yield relative to a corresponding control microbe that is not so genetically modified. For example, the oil yield of the genetically modified microbe can be increased by at least 10% relative to the corresponding control microbe. For example, the oil yield of the genetically modified microbe can be increased by at least 20%, 30%, 40%, 50%, or even more, relative to the corresponding control microbe.

In accordance with the method, the genetic modification can further cause at least one of an increase in malate dehydrogenase activity and an increase in ATP citrate lyase activity, as discussed above. Thus, in some examples, the genetic modification can cause both the increase in malate dehydrogenase activity and the increase in ATP citrate lyase activity.

In accordance with the method, the genetic modification can further cause at least one of a decrease in glyceraldehyde-3-phosphate dehydrogenase activity, an increase in fructose-1,6-bisphosphate aldolase activity, an increase in pyruvate kinase activity, and an increase in aconitase activity, as discussed above. Thus, in some examples, the genetic modification can cause at least two or at least three of the decrease in glyceraldehyde-3-phosphate dehydrogenase activity, the increase in fructose-1,6-bisphosphate aldolase activity, the increase in pyruvate kinase activity, and the increase in aconitase activity. Also in some examples, the genetic modification can cause all four of the decrease in glyceraldehyde-3-phosphate dehydrogenase activity, the increase in fructose-1,6-bisphosphate aldolase activity, the increase in pyruvate kinase activity, and the increase in aconitase activity.

In accordance with the method, at least one of the decreases in activity and at least one of the increases in activity can contribute to confer the increased oil yield of the genetically modified microbe, as discussed above.

The method can further comprise selecting the genetically modified microbe based on at least one of the decreases in activity and the increases in activity, as discussed above.

In accordance with the method, at least one of the decreases in activity can be based on a technique selected from the group consisting of mutagenesis. RNAi, expression of siRNA, gene silencing, homologous recombination, disruption of a regulatory sequence, partial gene deletion, and full gene deletion, as discussed above. Moreover, at least one of the increases in activity can be based on a technique selected from the group consisting of transformation, electroporation, transduction, introduction of recombinant DNA, introduction of a plasmid, and introduction of an artificial chromosome. In addition, at least one of the decreases in activity can be based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene. Furthermore, at least one of the increases in activity can be based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene.

As also noted above, also provided is a method of producing oil. The method can comprise obtaining a genetically modified microbe by the method for increasing oil yield, as described above. The method can also comprise extracting oil from the genetically modified microbe, e.g. by methods known in the art.

As also noted above, also provided is a genetically modified microbe that exhibits an increased oil yield relative to a corresponding control microbe that is not so genetically modified. The genetically modified microbe can be obtained by the method for increasing oil yield, as described above. The oil yield of the genetically modified microbe can be increased by at least 10% relative to the corresponding control microbe. For example, the oil yield of the genetically modified microbe can be increased by at least 20%, 30%, 40%, 50%, or even more, relative to the corresponding control microbe.

The following examples are for purposes of illustration and are not intended to limit the scope of the claims.

### Example 1

### Defining High-Yielding Oil Palms and Low-Yielding Oil Palms

Two screening populations of oil palm plants, a high-yielding screening population (also termed HY) and a low-yielding screening population (also termed LY), were used. The screening populations were derived from crosses of Serdang Avenue dura (at least 75% of Serdang Avenue dura) and AVROS pisifera (at least 75% of AVROS pisifera) to yield tenera progeny. The high-yielding and low-yielding palms are defined by the quantity of oil produced by them in tonnes per hectare per year. In this study, an oil palm was considered high-yielding if it produced more than 10 tonnes of oil per hectare per year, and an oil palm was considered low-yielding if it produced less than 6 tonnes of oil per hectare per year. Both screening populations were derived from a Carey Island oil palm plantation. The yield determinations were based on historical oil yield data for each sample.

For the examples that follow, samples of mesocarp tissue of individual plants selected from the high-yielding screening population and the low-yielding screening population were collected at specific time points of the fruit development. The mesocarp samples were sliced, pulverized in liquid nitrogen, and stored at −80° C. The pulverized mesocarp samples were then extracted and profiled using high-throughput omics platforms including transcriptomics, proteomics, and metabolomics to identify potential candidates/biomarkers (e.g. genes) related to yield trait, as follows.

### Example 2

### Arabidopsis Microarray Experiment

Arabidopsis One-Color Microarray-Based Gene Expression Analysis.

Samples of oil palm transcripts obtained from high-yielding palms and low yielding palms at 16 weeks after pollination were hybridized on 4×44K microarray formats. Procedures for the preparation, labeling of complex biological targets, and hybridization, washing, scanning, and feature extraction of Agilent's 60-mer oligonucleotide microarrays for gene expression analysis were adapted from “One-color microarray-based gene expression analysis version 6.0” by Agilent Technologies.

Results. The Arabidopsis microarray data showed triose-phosphate isomerase was down-regulated (>1.7 fold) in high-yielding palms compared to low-yielding palms at week 16 after pollination, with a p value of 0.02.

### Example 3

### 2-D DIGE Experiment

Preparation of Samples.

A modified protein extraction method published by He et al. (I) was used to isolate total mesocarp protein from oil palm fruitlets. Subsequently, the extracted protein samples were resuspended in 2-D cell lysis buffer (30 mM Tris-HCl, pH 8.8, containing 7 M urea, 2 M thiourea and 4% CHAPS). The mixture was sonicated at 4° C., followed by shaking for 30 minutes at room temperature. The samples were then centrifuged for 30 minutes at 14,000 rpm and the resulting supernatant was collected. Protein concentration of the supernatant fraction was measured using Bio-Rad protein assay method (Bradford, 1976). To aid downstream analysis, an internal standard (IS) was prepared, by mixing equal amount of protein from each sample, and included in the 2D-DIGE experiment.

CyDye Labeling.

For each sample, 30 μg of protein was mixed with 1.0 μl of diluted CyDye, and kept in dark on ice for 30 minute. Samples from each pair of high-yielding and low-yielding palms were labeled with Cy3 and Cy5, respectively, while the internal standard was labeled with Cy2. The labeling reaction was stopped by adding 1.0 μl of 10 mM Lysine to the sample, and incubating in dark on ice for additional 15 minutes. The 3 labeled samples were then mixed together. The 2×2-D Sample buffer (8 M urea, 4% CHAPS, 20 mg/ml DTT, 2% pharmalytes and truce amount of bromophenol blue), 100 μl Destreak solution and rehydration buffer (7 M urea, 2 M thiourea, 4% CHAPS, 20 mg/ml DTT, 1% pharmalytes and trace amount of bromophenol blue) were added to the labeling mix to make the total volume of 250 μl. The resulting mixture was thoroughly mixed and spun down prior to loading the labeled samples onto immobilized pH gradient gel (IPG) strips housed in a strip holder.

Set Up of 2D-DIGE Analytical Gels.

DIGE gels were designed to contain the appropriate sample pairings in order to facilitate gel analysis in the later part of the experiment. A total of 9 DIGE gels were produced with the sample pairing.

Internal Standard.

The internal standard (also termed IS) is used to match and normalize protein patterns across different DIGE gels, therefore negating the problem of inter-gel variation. It allows accurate quantification of differences between samples with an associated statistical significance. Quantitative comparisons of protein between samples are made on the relative change of each protein spot to its own in-gel internal standard.

IEF and SDS-PAGE.

After loading the labeled samples onto pH 4-7 IPG strips, isoelectric focusing (IEF) was run following the protocol provided by Amersham BioSciences (GE Healthcare, 2004). Upon finishing the IEF, the IPG strips were incubated in the freshly made equilibration buffer-1 (50 mM Tris-HCl, pH 8.8, containing 6 M urea, 30% glycerol, 2% SDS, trace amount of bromophenol blue and 10 mg/ml DTT) for 15 minutes with gentle shaking. The strips were then rinsed in the freshly made equilibration buffer-2 (50 mM Tris-HCl, pH 8.8, containing 6 M urea, 30% glycerol, 2% SDS, trace amount of bromophenol blue and 45 mg/ml DTT) for 10 minutes with gentle shaking. Following that, the IPG strips were rinsed in the SDS-polyacrylamide gel electrophoresis (SDS-PAGE) gel running buffer before transferring into 12% SDS-gels. The SDS-gels were run at 15° C. until running of the dye front out of the gels.

Image Scan and Data Analysis.

Gel images were scanned immediately following the SDS-PAGE using Typhoon TRIO (Amersham BioSciences) (data not shown). The scanned images were then analyzed by Image Quant software (version 6.0, Amersham BioSciences), followed by Biological Variation Analysis (BVA) using DeCyder™ 2D software version 6.5 (Amersham BioSciences). The protein spots that were found significantly being expressed differentially in at least 1.5 fold change were selected for mass spectrometry identification work using MALDI-ToF/ToF.

Results.

Based on 2D-DIGE analysis, three proteins that were involved in glycolysis pathway were determined to be differentially expressed between high-yielding and low-yielding palms across time points. These proteins are triose-phosphate isomerase, which is expressed at lower levels in high-yielding versus low-yielding palms, fructose bisphosphate aldolase, which is expressed at higher levels in high-yielding versus low-yielding palms, and glyceraldehyde-3-phosphate dehydrogenase, which is expressed at lower levels in high-yielding versus low-yielding palms.

### Example 4

### Dot-Blot Immunoassay

Protein Extraction for Dot-Blot Immunoassay Validation.

TCA extraction buffer (pre-cooled at −20° C., 0.2 g+0.5 mL) was added to fine powder and further ground using mini plastic grinder. The mixture was mixed and mashed well before adding another 1 mL buffer and samples were incubated at −20° C. for 1 hour. The samples were then centrifuged at max speed (13.2 g) at 4° C. for 15 min. Sample tubes were kept on ice and the supernatant was removed using a pipette. Approximately 1.8 ml wash buffer was added to the sample and pellets were re-suspended and/or crushed using pipette tips. Samples were incubated at −20° C. for 1 hour and centrifuged at max speed at 4° C. for 15 min. Supernatant was removed and the washing step was repeated for a total of three times. Sample powder was air-dried on ice for 30 min and re-suspended in 500 μl of lysis/USB buffer. The solution was incubated at 37° C. for 1 hour with continuous shaking. Samples were centrifuged at max speed at room temperature for 15 min and the supernatant was transferred to clean tubes before storing at −80° C. (pellet was stored at this temperature as back up for further use). To further elute protein from pellet, an additional 500 μl of lysis/USB buffer was added to samples, which were then shaken at room temperature for 1 hour. The back-up supernatant was transferred to clean microcentrifuge tubes and stored at −80° C.

Protein Quantification (Bradford Assay).

Protein stocks (1.4 μg/μl) that were diluted five-fold were used to construct the standard curve for quantification. Sample concentrations ranged from 0.244 μg/μl (lowest) to 2.934 μg/μl (highest). Upon obtaining the protein stock concentration, a working stock (330 μl) was prepared at a final concentration of 0.2 μg/μl using PBS buffer (+10% glycerol) for dot-blotting onto membranes.

Dot-Blot Array Using 386 Pin Replicator.

Samples were prepared at concentrations of 0.20 μg/μl and 0.02 μg/(10× dilution), respectively. Using a replicator, the proteins were applied onto the membrane and fan-dried after each application. The procedure was repeated for a total of 5 rounds (equivalent to stamping 0.20 μg or 0.02 μg of protein on each spot since replicator pins deliver 0.2 μl of sample). Membranes were allowed to air dry overnight. Membranes were then removed from plates and cut down to size. Membranes were kept in between the original protective paper and stored in air-tight containers in dry environment until further use.

Preparation for Screening.

Individual membranes were clipped onto glass slides, two on each slide, back facing inwards. The clipped membranes were dipped into a container filled with cold 0.1% PBS-T (pH 7.4) and stirred on a magnetic stirrer for 40 min. The 0.1% PBS-T was replaced with cold 0.05% PBS-T and washed continuously for 15 min. The solution was replaced with fresh cold 0.05% PBS-T and stirred continuously for 7 min and the step above was repeated.

Antibody Incubations.

Approximately 1 ml of PBS-T 0.05% was pipetted onto blank membranes and 1 ml diluted antibody was pipetted onto corresponding membranes in incubation containers. The membranes were incubated overnight on a Belly Dancer brand laboratory shaker (IBI Scientific) at 4° C. or room temperature for 2 to 3 hours depending on optimized conditions for individual antibodies. The used sera were retained for further experiments or discarded into a bottle to be autoclaved. The membranes were clipped onto glass slides and washed in cold 0.05% PBS-T for 15 min, followed by 7 min twice using fresh 0.05% PBS-T. The membranes were laid back into clean incubation containers in a manner to ensure that no bubbles were trapped underneath the membranes. Diluted secondary antibody (1 mL) was added to each membrane. For secondary antibodies with background signals, pre-adsorption was performed with 1% BSA, followed by shaking at 37° C. for 2 hours. The containers were covered and incubated for 2.5 hours on a Belly Dancer laboratory shaker at 4° C. or room temperature. The secondary antibody was discarded and washing steps were repeated as in steps above for 15 min, 7 min, and 7 min, each round with fresh, cold 0.05% PBS-T.

Development and Documentation.

The membranes were laid back in incubation containers, again in a manner to ensure that no bubbles were trapped underneath the membranes, and any remaining 0.05% PBS-T was removed. Fresh NBT/BCIP was prepared according to manufacturer guidelines using alkaline phosphatase (AP) buffer (100 mM Tris [pH 9.0], 150 mM NaCl, 1 mM MgCl₂). NBT/BCIP solution (1.5 mL) was added per membrane, followed by incubation on a Belly Dancer laboratory shaker until purple color is developed (approximately 30-45 min). The reaction was then stopped and membranes were soaked in water. The developed membranes were scanned and processed using Adobe Photoshop C84 Extended and Olympus Micro software to automatically capture and transform spot densities into excel sheets.

Results.

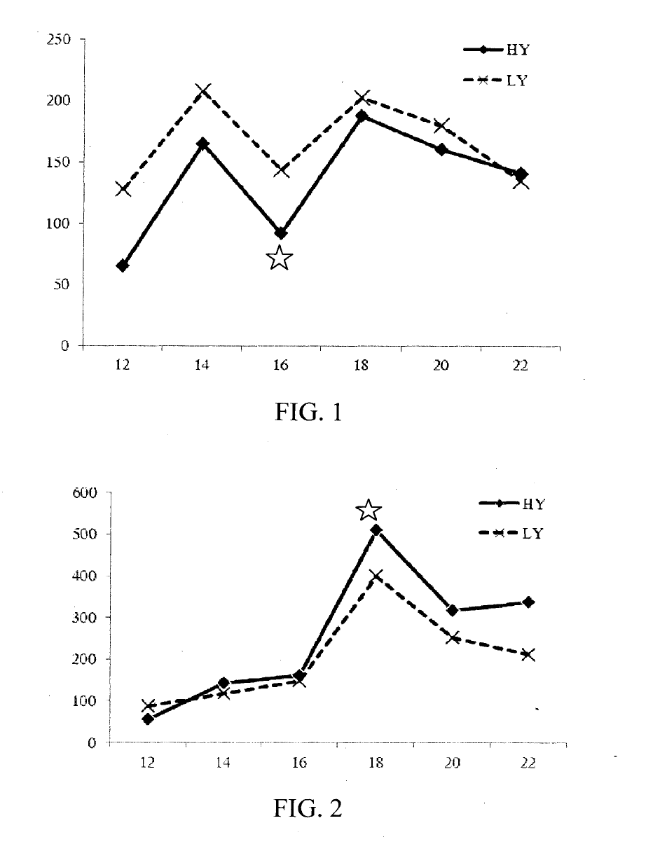

The dot blot immunoassay indicated that triose-phosphate isomerase expression was lower in high-yielding palms than in low-yielding palms at week 16 after pollination (FIG. 1) and that fructose bisphosphate aldolase expression was higher in high-yielding palms than in low-yielding palms at week 18 after pollination (FIG. 2).

### Example 5

### Transcriptional Expression Analyses

Experimental Setup and Sampling.

Eight biological samples were selected respectively from high and low yielding populations based on the average yearly performance of oil content in the mesocarp tissue of the oil palm. Inflorescences of the selected palms were open pollinated in the field. Each pollinated inflorescence was kept until a specific time point according to the pollination date thereof, specifically 12, 14, 16, 18, 20, and 22 weeks after pollination. The fresh fruit bunches of selected palms were harvested at the specific time points and mesocarp tissues were obtained from harvested fruit bunches.

Oil Palm Mesocarp RNA Extraction.

Total RNA was extracted from oil palm mesocarp tissue of six different time points as described above by using a modified RNA extraction method by Asemota et al. (2). The modification includes use of a composition of extraction buffer to which no phenol was added. The modified protocol also included an extra step of chloroform cleanup before LiCl precipitation. The concentrations and purity of total RNA were determined by quantification with a NanoDrop brand spectrophotometer (Thermo Scientific). The AU 260/280 and AU 260/230 were measured and samples with ratios of 1.8-2.0 were accepted in quality. Gel electrophoresis was also performed on 1 μg of total RNA by using 1% agarose gel in TAE buffer to further determine the RNA quality. Gels were imaged by use of Alphalmager 2200 brand imaging system (Alpha Innotech).

Custom Design Oil Palm Mesocarp Agilent Array.

The transcriptional expression of high and low yielding oil palm populations throughout the six different time points were determined by hybridizing the samples on custom oil palm mesocarp gene expression arrays. Customized gene expression oil palm mesocarp arrays were designed based on an Agilent microarray platform in 2×105K format. The probes were designed based on 31794 sequences from mesocarp transcriptome sequencing whereby the annotations were obtained by comparing to the UniProt database (unpublished data). Probes were designed using Agilent internal designed program through Agilent's eArray website. The transcriptome sequences were represented by three different probes which covered different parts of the transcriptome sequences.

Synthesis of cRNA, Microarray Hybridization, and Scanning.

The total RNA samples from the mesocarp were treated and labeled with one-color Cy3 dye according to Agilent's Low Input Quick Amp Labeling protocol version 6.0 (3) as follows. A total of 100 ng of total RNA was used to synthesize cRNA, which was labeled with Cy3 dye. Labeled cRNAs were used to hybridize on the mesocarp array at 65° C. for 16 hours. After hybridization, the mesocarp array was subjected to two steps of washing with wash buffer 1 and 2 for 1 minute for both washing steps. The array was air-dried for a few seconds before the image was scanned by using an Agilent B scanner.

Data Extraction, Analysis, Selection of Candidates, and Classification.

Raw microarray data were extracted from scanned images by using Feature Extraction software version 10.7.31 from Agilent. After the extraction, raw microarray data from each time point were further analyzed using R software. In the analysis of R software, the raw microarray data were normalized using a Quantile normalization algorithm. After normalization, each probe signal value from high yielding population samples was compared to probe signal value of samples from low yielding population, to obtain the relative gene expression ratios of each probe between high and low yielding populations. Probes with expression ratios of greater than 1.5 fold were selected. The differentially expressed candidates (probes) were further filtered by using the corresponding p-value obtained. The p-value cut-off for each time point was based on the distribution of general p-value for all probes in that particular time point where non-differentially expressed candidates were equally distributed, in accordance with Fodor et al. (4). In this experiment, the cut-off p-values of weeks 12, 14, 16, 18, and 20 were 0.05 and the cut-off p-value for week 22 was 0.01. The differentially expressed candidates from each time point were classified into different categories according to their gene functions using WEGO Gene Ontology (GO) system.

Results and Discussion.

A total of 2116 candidate differentially expressed probes were obtained from six different time points by applying the cut-off above. The candidate differentially expressed probes were distributed as follows: week 12 (100 probes); week 14 (270 probes); week 16 (134 probes); week 18 (171 probes); week 20 (588 probes); and week 22 (853 probes). All 2116 candidates probes represent specific sequences, termed isotigs, which can be used to design probes on arrays.

Annotation of the candidate probes was carried out based on the Swiss-Prot database. Annotations were distributed as follows: week 12 (56 annotations from the 100 probes); week 14 (180 annotations from the 270 probes); week 16 (93 annotations from the 134 probes); week 18 (108 annotations from the 171 probes); week 20 (370 annotations from the 588 probes); and week 22 (524 annotations from the 853 probes). In total, 1331 annotated candidate probes were obtained from the six time points. The Blast2GO program was used to obtain Gene Ontology (GO) annotations for all annotated candidate probes. The annotated candidate probes were further classified according to GO using a WEGO web tool of Ye et al. (5) and cluster of orthologous group as mentioned in Wang et al. (6).

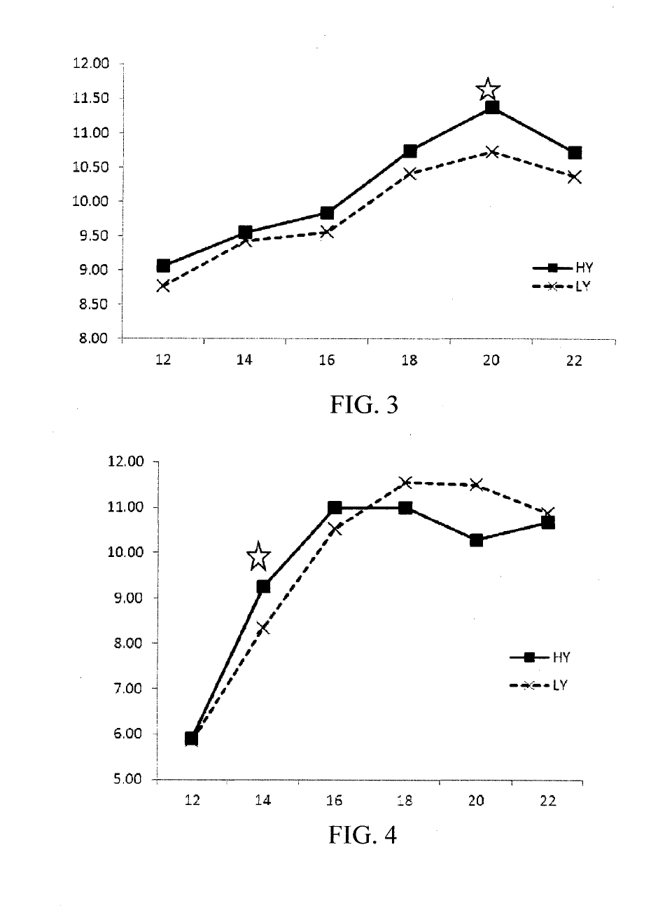

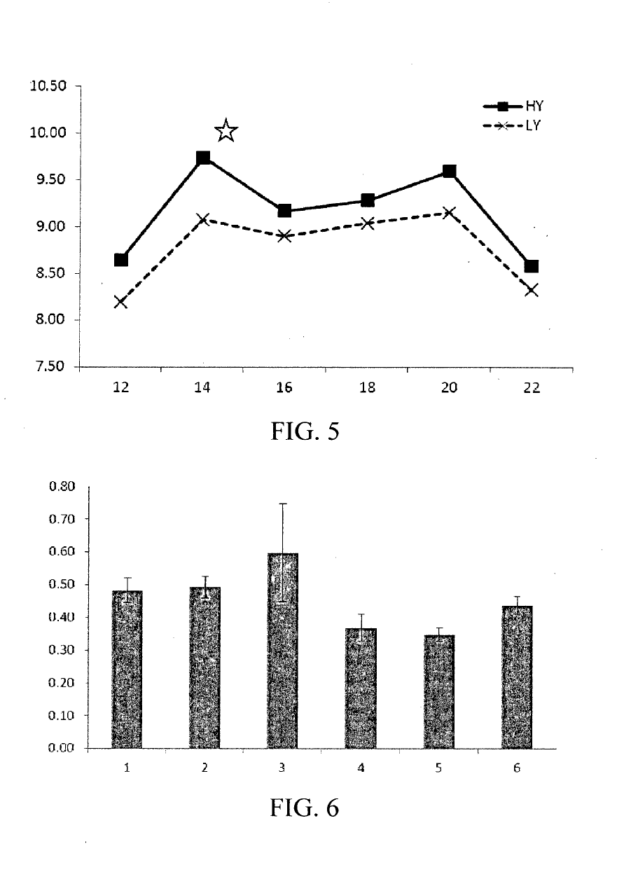

In accordance with the annotations, the transcriptional expression data indicated that ATP citrate lyase expression was higher in high-yielding palms than in low-yielding palms at week 20 after pollination (FIG. 3), that pyruvate kinase expression was higher in high-yielding palms than in low-yielding palms at week 14 after pollination (FIG. 4), and that aconitase expression was higher in the high-yielding palms than in low-yielding palms at 14 weeks after pollination (FIG. 5).

### Example 6

### Mass Spectrometry Profiling

Capillary Electrophoresis-Mass Spectrometry Method.

Mesocarp samples (55 to 70 mg) were added to 500 μl of methanol including 50 M of internal standard (Methionine sulfone and D-camphor-10-sulfonic acid) in the tubes and were frozen with liquid nitrogen. They were homogenized using a cell breakage machine with beads (TOMY, MS-100R). Chloroform (500 μl) and Milli-Q water (200 μl) were added to the homogenates, followed by thorough mixing and then centrifugation (2,300×g, 4° C., 5 min). After centrifugation, the resulting water layer (400 μl) was harvested and filtered with a 5-kDa cut-off filter (MILLIPORE, Ultrafree MC UFC3LCC). The filtrates were desiccated and then dissolved with 50 μl of Milli-Q water. Judging from peak shapes and intensities, they were diluted by 50% and 20% for CE-TOFMS analysis in cation and anion modes, respectively.

Samples were analyzed using an Agilent CE-TOFMS system (Agilent Technologies) equipped with a fused silica capillary (i.d. 50 μm×80 cm). For cation mode, the run buffer was Cation Buffer Solution (p/n: H3301-1001), and the rinse buffer was Cation Buffer Solution (p/n: H3301-1001). Sample injection pressure was 50 mbar (10 sec). The MS parameters were as follows: CE voltage: Positive, 27 kV; MS ionization: ESI Positive; MS capillary voltage: 4,000 V; MS scan range: m/z 50-1,000; Sheath liquid: HMT Sheath liquid (p/n: H3301-1020). For anion mode, the run buffer was Anion Buffer Solution (p/n: H3302-1021), and the rinse buffer was Anion Buffer Solution (p/n: H3302-1022). Sample injection pressure was 50 mbar (25 sec). The MS parameters were as follows: CE voltage: Positive, 30 kV; MS ionization: ESI Negative; MS capillary voltage: 3,500 V; MS scan range: m/z 50-1,000; Sheath liquid: HMT Sheath Liquid (p/n: H3301-1020).

Data Acquisition and Analysis.

Peaks detected in CE-TOFMS analysis were extracted using the automatic integration software (MasterHands ver.2.1.0.1). Peak information including m/z, migration time (MT) and area was obtained. Peak area was converted into relative peak area according to the following equation: Relative Peak Area=(Metabolite Peak Area)/(Internal Standard Peak Area×Sample Amount). Principle component analysis (PCA) and Orthogonal Partial Least Square-Discrimination Analysis (OPLS-DA) were used to identify metabolites that reflect the differences between high and low yielding palm trees. PCA and OPLS-DA were performed with SPSS ver. 18 and SIMCA-P ver. 12. Quantitative estimation was performed using 107 metabolites, including intermediates in glycolysis, TCA cycle, amino acids, and nucleic acids. Concentrations of the metabolites were calculated by normalization of the peak area of an internal standard. Standard curves for each of the metabolites were obtained by single-point at 100 μM of standard metabolites.

Results.

Metabolites such as 2-phosphoglyceric acid, 3-phosphoglyceric acid, and fructose-1,6-bisphosphate were found to be in low concentration in high-yielding palms. These metabolites are highly consumed or have been utilized for oil biosynthesis in the mesocarps of high-yielding palms. It was found that the concentration of glycerol-3-phosphate was higher in high-yielding palms from week 16 to matured stage. This indicates that a reduced triose-phosphate isomerase activity and an increased glycerol-3-phosphate dehydrogenase activity enhanced production of dihydroxyacetone phosphate, which was converted into glycerol-3-phosphate, providing building blocks for oil biosynthesis.

### Example 7

### Functional Characterization of Glycolytic Genes

Model systems.

Functional characterization of oil palm genes of glycolysis and lipid biosynthesis can be carried out using transformation-based approaches in model plant and microbial systems such as Arabidopsis sp. and yeast. Changes in levels of metabolites, lipid content, protein expression, or other phenotypic changes can be compared between control (i.e. wild-type) plants or microbes, mutants that over-express a particular gene, and mutants that are complemented for a particular gene. SNP analysis and function prediction of genes related to the oil palm glycolytic pathway can be carried out and the SNPs effects on these genes can be studied in yeast.

Experimental Design for Oil Palm Gene Over-Expression Study in Yeast.

The following approach was used to characterize the effect of over-expression of four oil palm genes, specifically triose-phosphate isomerase, fructose-1,6-bisphosphate aldolase, glycerol-3-phosphate dehydrogenase, and glyceraldehyde-3-phosphate dehydrogenase, in yeast. Each of the four genes was isolated from DNA of oil palm mesocarp. The sequence of each gene was verified. Each gene of interest was sub-cloned into pYES2.1 TOPO vector. Sequence verification was carried out to check the orientation of the cloned gene in the vector. A yeast strain was transformed with the vector. Yeast transformants were verified using a PCR approach. Metabolite profiling and analysis was then carried out as follows.

Yeast Strain and Growth Conditions.

Saccharomyces cerevisiae cells were grown in synthetic complete (SC) minimal agar medium supplemented with appropriate auxotrophic supplements at 30° C. for 2 days. The yeast strain was grown overnight in SC minimal broth medium (without Uracil in the case of cultures of over-expressed mutants) at 30° C., 200 rpm. The overnight culture was centrifuged at 7500 rpm at ambient temperature for 2 min. The supernatant was discarded and replenished with fresh broth medium. Initial OD₆₀₀ was diluted to 0.5 and inoculated to fresh liquid medium. The yeast cultures (3 replicates) were harvested at 6 hours after incubation and OD₆₀₀ was adjusted to 1.0 for extraction.

Samples for the Intracellular Metabolites (Quenching).

Samples of 5 mL (OD₆₀₀ fixed to 1.0) were harvested at 6 hours and centrifuged for 2 min at 7500 rpm, 4° C. Each supernatant was discarded and the corresponding cell pellet was re-suspended with 5 mL fresh medium. A quenching method was adapted from Castrillo et al. (7). In accordance with the method, 10 mL of 60% (v/v) buffered MeOH:tricine (10 mM, pH 7.4) at −40° C. was added to each sample, the mixture quickly vortexed (about 1 second) and incubated at −50° C. for about 5 min. Supernatants were separated by centrifugation (7500 rpm, 5 min, −9° C., adaptors pre-cooled at −40° C.). The quenching was performed in quadruplicate samples.

Boiling Ethanol Extraction.

An extraction method using buffered ethanol:ammonium acetate (10 mM, pH 7.5) was adapted from Ewald et al. (8). Extraction was performed in 50 mL Falcon tubes (2 replicates). Tubes containing 5 mL of 75% (v/v) buffered ethanol:ammonium acetate (10 mM, pH 7.5) and 0.4 mg/mL ribitol were pre-heated in a water bath at 80° C. Samples were added to the boiling buffered ethanol, immediately mixed, and placed in water at 80° C. for 3 min. Samples were centrifuged for 2 min at 7500 rpm to remove biomass debris. Samples were then filtered through 0.2 μm filter, freeze-dried and stored at −80° C. prior to LC-MS analysis.

Ultra Performance Liquid Chromatography.

Solvents used for liquid chromatography were of Optima LC-MS grade (Fisher Scientific, Fair Lawn, N.J., USA). Millipore purified water was used in the preparation of standards and sample solutions. The standards of sugar phosphates/glycolytic-related compounds were of analytical grade and obtained from Sigma (St. Louis. Mo., USA). An Acquity UPLC system coupled to a detector Xevo TQs was used for analytical determination of the targeted metabolites in the mesocarp sample. Chromatography was conducted with an Amide (2.1×100 mm, 1.7 μm) column. Column and sample were maintained at 35° C. and 4° C., respectively. The injection volume was 3 μL with partial loop injection. The mobile phase A consisted of 10 mM ammonium formate, pH 9.0, while mobile phase B was 90% (v/v) acetonitrile in 10 mM ammonium formate, pH 9.0. A linear gradient was used as follows: 20% to 50% B from initial time to 5 min; 50% B from 5 min to 8 min; 50% to 20% B from 8 min to 10 min; 20% B from 10 min to 15 min. The total run time was 15 min, the flow was constant at 0.3 mL/min, and the curves for gradients were each 6.

Mass Spectrometry.

The mass spectrometry was operated in both positive and negative mode with multiple reaction monitoring using ESI. The capillary voltage was set at 3.5 kV, desolvation gas set at 800 L/hr at temperature of 300° C. The collision gas flow was set at 0.15 mL/min. The MRM settings in the MS/MS function with corresponding cone voltage and collision energy were optimized for each standard compound. Auto dwell times were set for positive mode and negative mode, respectively. Total acquisition durations for both UPLC and MS were set at 15 min and 5 min, respectively. Data were acquired and processed using MassLynx V4.1 and TargetLynx, respectively. The glycolytic metabolites determined were fructose-1,6-bisphosphate; dihydroxyacetone phosphate; glyceraldehyde-3-phosphate; glycerol-3-phosphate; 3-phosphoglyceric acid; phosphoenolpyruvate; and ribitol (as internal standard).

Total Lipid Extraction.

Freeze-dried samples of 50 mg were extracted with chloroform:methanol:water (2 mL:2 mL:2 mL), vortexed for 30 seconds, and centrifuged at 4000 rpm, 4° C. for 10 min. The chloroform layer was collected in a new pre-weighed tube. The samples were re-extracted twice with 2 mL of chloroform, vortexed, and centrifuged. The chloroform extracts (second and third extractions) were combined with the first chloroform extract and dried. The weight of dried extracts (lipid content) were measured.

Results: Differential Levels of Metabolites in Overexpressed Transformants.

Overexpression of oil palm fructose bisphosphate aldolase in yeast resulted in intracellular fructose bisphosphate levels that were much lower than for the wild-type yeast. Fructose bisphosphate aldolase stimulated the metabolism of fructose bisphosphate to dihydroxyacetone phosphate (9 fold) and glyceraldehyde 3-phosphate (11 fold), which greatly decreased the fructose bisphosphate pool. High fructose bisphosphate aldolase activity also results in accumulation of other metabolites such as glycerol 3-phosphate, 3-phosphoglyceric acid, and phosphoenolpyruvate. Thus, the oil palm fructose bisphosphate aldolase showed functionality in directing flux towards dihydroxyacetone phosphate and glyceraldehyde 3-phosphate production.

Overexpression of oil-palm cytosolic triose-phosphate isomerase, which catalyzes interconversion of dihydroxyacetone phosphate into glyceraldehyde 3-phosphate reversibly in yeast resulted in an increase of glyceraldehyde 3-phosphate and dihydroxyacetone phosphate. Accumulation of 3-phosphoglyceric acid was observed as glyceraldehyde 3-phosphate was rapidly converted to 3-phosphoglyceric acid by glyceraldehyde-3-phosphate dehydrogenase in the next step of glycolysis. Thus, the oil palm cytosolic triose-phosphate isomerase showed functionality in directing flux towards glyceraldehyde 3-phosphate and 3-phosphoglyceric acid production.

Overexpression of glyceraldehyde-3-phosphate dehydrogenase resulted in a decreased level of glyceraldehyde 3-phosphate, while an accumulation of 3-phosphoglyceric acid (>15 fold) was observed in the transformants in comparison to wild-type. Glyceraldehyde-3-phosphate dehydrogenase is essential for the maintenance of cellular ATP levels and carbohydrate metabolism (Rius et al. (9)). Thus, the oil palm glyceraldehyde-3-phosphate dehydrogenase showed functionality in directing flux towards 3-phosphoglyceric acid production.

Overexpression of glycerol-3-phosphate dehydrogenase in yeast resulted in differential levels of glycolytic metabolites in comparison to wild-type yeast. Glycerol-3-phosphate dehydrogenase catalyzes the reduction of dihydroxyacetone phosphate to glycerol 3-phosphate. An increase in conversion of glycerol 3-phosphate from dihydroxyacetone phosphate was observed in the overexpression of glycerol-3-phosphate dehydrogenase transformants. Glycerol 3-phosphate and dihydroxyacetone phosphate can serve as precursors to synthesize phospholipids and glycerolipids (Wang et al. (10)). Thus, the oil palm glycerol-3-phosphate dehydrogenase showed functionality in directing flux towards glycerol 3-phosphate production. Overexpression of glycerol-3-phosphate dehydrogenase in yeast diverts the carbon flux towards glycerol, but results in poor growth rate due to cytotoxic effect of acetaldehyde accumulation (Remize et al. (11)). Results on the biomass growth showed that there was a decrease in dry biomass weight of about 30% in yeast over-expressing glycerol-3-phosphate dehydrogenase compared to wild-type yeast.

Total Lipid Contents of the Overexpression Glycolytic Genes in Yeast.

Total lipid contents of yeast transformants and wild-type yeast are shown (FIG. 6). Overexpression of fructose bisphosphate aldolase and glycerol-3-phosphate dehydrogenase leads to an increase of lipid contents of 10% and 33%, respectively. Moreover, overexpression of triose-phosphate isomerase and glyceraldehyde-3-phosphate dehydrogenase resulted in a decrease of lipid contents of 59% and 21%, respectively. In addition, it is predicted that reduced expression and/or activity of triose-phosphate isomerase and glyceraldehyde-3-phosphate dehydrogenase would result in an increase in lipid contents.

### Example 8

### Additional Metabolite Studies in High-Yielding and Low-Yielding Oil Palms

Additional comparisons of differences in metabolite concentrations in high-yielding (HY) palms versus low-yielding (LY) palms were carried out. Specifically, eight palms were selected from each of the high- and low-yielding screening populations essentially as described in Example 1. The high-yielding population was previously determined to yield 10 to 12 tonnes of palm oil per hectare per year, and a low-yielding population was previously determined to yield 4 to 7 tonnes of palm oil per hectare per year. Fruits were harvested from plants at 12, 14, 16, 18, 20, and 22 weeks after pollination to represent fruit development before, during, and after lipid biosynthesis and ripening occurs. Profiling of the two populations was carried out using CE-MS with the same extraction and acquisition parameters described in Example 6. Secondary profiling was carried out using LC-MS (LTQ-Orbitrap and LC-triple quadrupole MS) as well as GC-MS in order to investigate the instrument-specificity of the results, as follows.

LC-MS (LTQ-Orbitrap) Method:

Extraction for LC-MS (LTQ-Orbitrap) profiling was carried out as follows. Mesocarp tissue from each fruit was lyophilized. Approximately 100 mg of each lyophilized mesocarp tissue was weighed into a 15 mL FALCON tube. Then 2 mL of 75% (v/v) isopropanol with 0.01% BHT was added to the tissue. The mixture was heated and shaken in a thermo-mixer for 15 minutes at 750 rpm. A mixture of (i) 0.2 mg/mL phenanthrene dissolved in 2 mL chloroform, (ii) 0.2 mg/mL ribitol dissolved in methanol, and (iii) 1.6 mL water was used for internal standards for lipids and polar metabolites. The mixture was shaken for 30 seconds using a vortex mixer. Incubation was carried out for 30 minutes at 250 rpm and 4° C. in a thermo-mixer. Then 4 mL of chloroform and water at a ratio 1:1 was added to the mixture. The mixture was shaken 30 seconds using a vortex mixer. The mixture was then centrifuged at 4° C. and 4000 rpm for 10 minutes to separate the lipid layer (in chloroform, at the bottom) and the polar layer (in methanol and water, at the top).

LC-MS (LTQ-Orbitrap) profiling was conducted on the polar (top) layer as follows. LC-MS data were acquired using Accela-LTQ Orbitrap brand instrument (Thermo Fisher, Germany). Sample analysis was carried out in positive and negative ion modes of detection. The mass scanning range was 100 to 2000 m/z, while capillary temperature was 300° C. and sheath gas auxiliary gas flow rates were 35 and 15 arbitrary units (“arb”), respectively. The sweep gas flow rate was set at 1 arb I-spray voltage at 4.5 kb. The resolution was 30,000 at 1 microscan and maximum injection time at 500 ms. The capillary voltage and tube lens were set at 40 V and 80 V, respectively for positive ion modes. Both respective parameters were set at −2.00 V and −47.44 V for negative ion mode. The MS/MS spectra of metabolites were obtained by collision energy ramp at 35 V. Autosampler temperature was set at 10° C. with 3.0 μL injection volume. The LC/MS system (controlled by Xcalibur brand software version 2.0, Thermo Fisher Corporation) was run in binary gradient mode. Solvent A was 0.1% v/v formic acid/water and solvent B was acetonitrile containing 0.1% v/v formic acid. The flow rate was 0.2 mL/min. An Acquity UPLC HSS T3 chromatography column (1.8 μm, 2.1×100 mm; Waters, Malaysia) set at 45° C. was used for analyses. The gradient was as follows: 1% B (0-1.8 min), 10% B (3 min) to 40% B at 20 min and hold for 3 min, 90% B at 26-28 min and 1% B at 29-35 min. The raw data were processed and compared using Sieve version 1.2 (Thermo Fisher, Alpha Analytical, Malaysia) with the frame time and m/z width set at 1.5 min and 0.002, respectively.

LC-MS (Xevo Triple Quad) Method:

Mesocarp sample extraction was performed in the same manner as used for LC-MS (LTQ-Orbitrap) analysis. An Acquity UPLC system coupled to a detector Xevo TQs was used for analytical determination of the targeted metabolites in the mesocarp samples. Chromatography was conducted with an HSS T3 (2.1×100 mm, 1.7 μm) column. The column and samples were maintained at 45° C. and 4° C., respectively. The LC mobile phases used were 0.1% formic acid in water (solvent A) and 0.1% formic acid in acetonitrile (solvent B). The flow rate was 0.3 mL/min. The elution gradient was as follows: initial hold at 95% solvent A; 0 to 3 min linear gradient to 60% solvent A; 3 to 5 min 5% solvent A; 5 to 5.1 min linear gradient to 95% solvent A and hold on to 7 min. Injection volume was 3 μL.

The mass spectrometry was operated in both positive and negative mode with multiple reaction monitoring using ESI. The capillary voltage was set at 2.9 kV, and desolvation gas was set at 800 L/hr at temperature of 350° C. The collision gas flow was set at 0.15 mL/min. The MRM settings in the MS/MS function with corresponding cone voltage and collision energy were optimized for each standard compound. Auto dwell times were set for positive mode and negative mode, respectively. Total acquisition duration for both UPLC and MS was set at 15 min. Data were acquired and processed using MassLynx V4.1 and TargetLynx, respectively.

Standard compounds were weighed and dissolved with 5% (v/v) acetonitrile in MilliQ water to make a stock solution with final concentration of 1 mg/mL (1000 ppm). The stock solutions were diluted to 1 ppm as working stock solutions. A mix standard at concentration of 1 ppm was prepared and injected into LC-MS daily to check system sensitivity and reproducibility.

GC-MS Method:

Mesocarp sample extraction was performed in the same manner as used for LC-MS (LTQ-Orbitrap) analysis. Derivatization of the polar metabolites was then conducted to enable detection using GC-MS. Samples were taken out from storage and placed in a SpeedVac concentrator for 30 minutes prior to derivatization. This was to ensure that all water content is dried off as moisture will hinder the derivatization process. Then 120 μL of 20 mg/mL methoxyamine hydrochloride in pyridine was added and samples incubated at 60° C. for 4 hours. Following this, 120 μL of N-methyl-N-(trimethylsilyl)trifluoroacetamide (MSTFA) with 1% trimethylchlorosilane was added. This was followed with further incubation at 60° C. for 1 hour. Derivatized samples were then analyzed using an Agilent 6890N Gas Chromatograph coupled with an Agilent 5973i Mass Detector and 6890 series autosampler. Volatiles were separated on a DB-5 ms column, 30 m×0.25 mm i.d.×0.25 μm film thickness. The temperature programme was set at 80° C., held for 3 minutes, followed by a 5° C. per minute ramp to 315° C., and holding at this temperature for 5 minutes. The injection port was splitless at 280° C. while column flow was maintained constant at 1 mL/min of Helium gas. The MS source was set at 230° C. with a scanning range of m/z 50 to m/z 650. Sample of 1 μL was injected into a programmable injector. The total run time is 55 minutes.

Results:

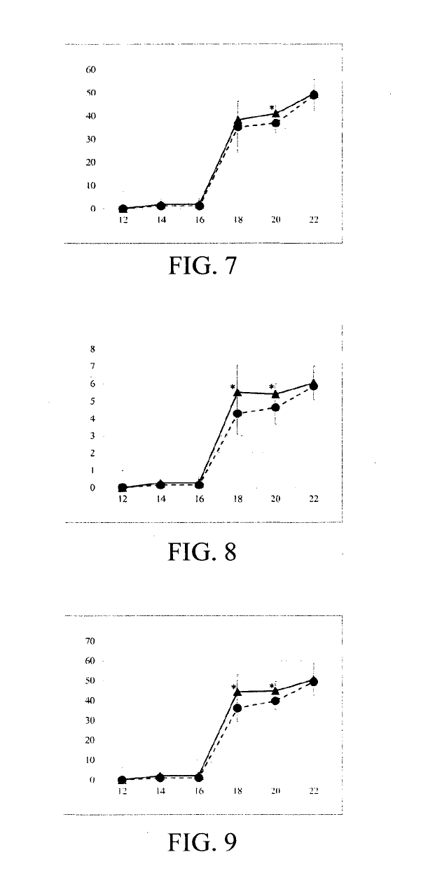

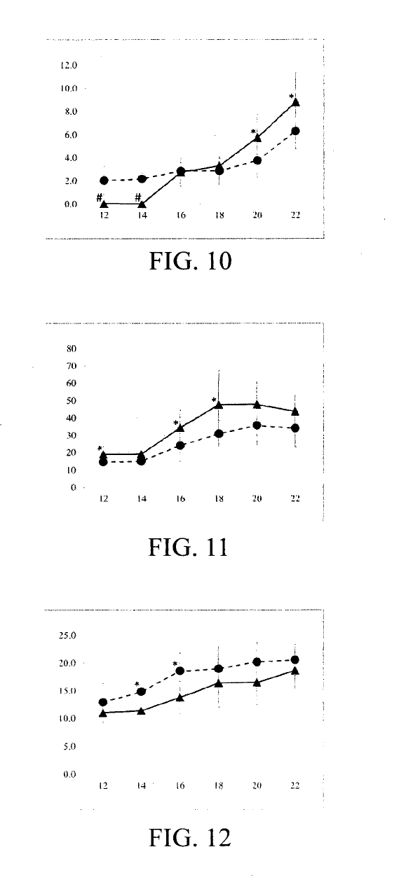

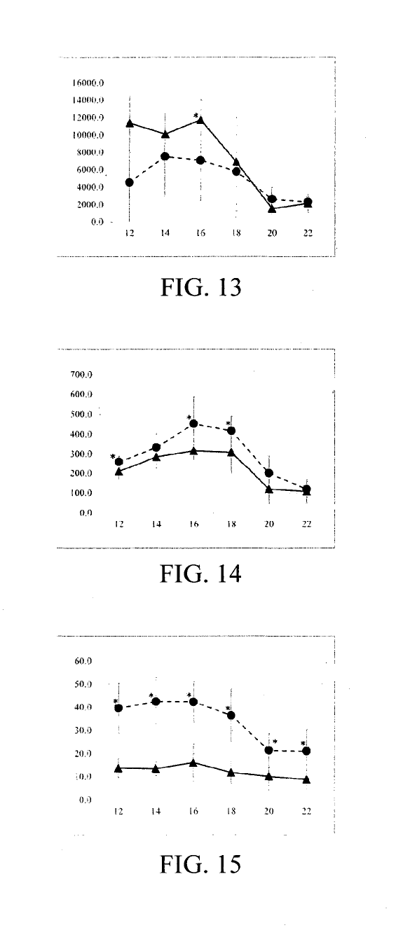

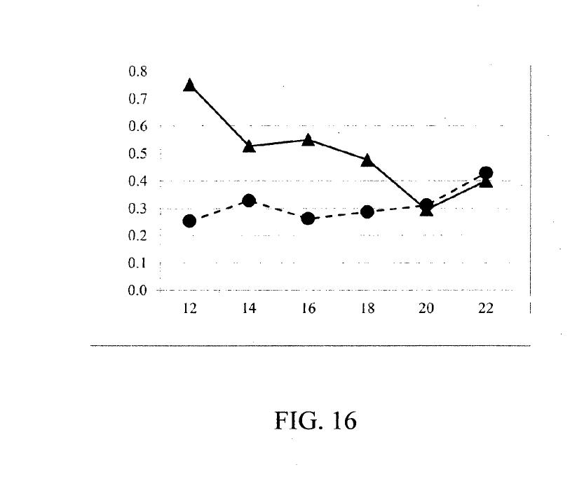

Data from primary and secondary profiling were analyzed as described in Example 6. Results indicating relative metabolite concentrations versus time (12 to 22 weeks after pollination) for mesocarp tissue of fruits of high-yielding oil palm plants (triangles) versus low-yielding oil palm plants (circles) are shown for 16:0 fatty acids (FIG. 7), 18:0 fatty acids (FIG. 8), 18:1 fatty acids (FIG. 9), fructose 1,6-bisphosphate (FIG. 10), glycerol 3-phosphate (FIG. 11), 3-phosphoglyceric acid (FIG. 12), malic acid (FIG. 13), isocitric acid (FIG. 14), and 2-oxoglutaric acid (FIG. 15). Ratios of malic acid to citric acid versus time (12 to 22 weeks after pollination), for mesocarp tissue of fruits of high-yielding oil palm plants (triangles) versus low-yielding oil palm plants (circles) are also shown (FIG. 16).

As can be seen, citric acid (FIG. 16), isocitric acid (FIG. 14) and, more markedly, 2-oxoglutaric acid (FIG. 15) all appeared at lower concentrations in the HY group of palms just preceding and during the early stages of lipid biosynthesis (14-18 weeks after pollination). In contrast, malic acid (FIGS. 13 and 16) exhibited a very different concentration profile between the HY and LY groups, maintaining a significantly higher concentration in HY palms from 12 weeks after pollination through to the mid-point of lipid biosynthesis (18 weeks after pollination) when it dropped to a level similar to that of the LY group. All of the organic acids in the TCA cycle were at their lowest concentrations during the final stage of fruit maturation. The differing trends of malic acid and citric acid concentration in HY and LY palms can be seen (FIG. 16), where the ratio of malic acid to citric acid is higher in HY palms from 12 weeks after pollination until when lipid biosynthesis reaches a maximum at 16-18 weeks after pollination, at which time the ratio decreases steadily to the same as LY palms. The higher consumption rate of malic acid during peak lipid production in HY palms indicates higher malic acid dehydrogenase activity. The relatively low concentrations of isocitric acid and 2-oxoglutaric acid observed in HY palms during lipid biosynthesis is likely to result from higher utilization of acetyl-CoA for fatty acid production and possibly of other major biosynthetic precursors leading to amino acid/protein production.

As can also be seen, the results show that there is not a deleterious shift in the profile of fatty acids C16:0 (FIG. 7), C18:0 (FIG. 8), or C18:1 (FIG. 9) of the oil of the HY palms in comparison to the oil of the LY palms.

The results indicate a divergence in carbon flux away from pyruvate and towards glycerol-3-phosphate as well as significant increases in the malate to citrate ratio preceding and during the lipid biosynthesis periods of fruit development. Simultaneous increases in production of glycerol-3-phosphate through glycolysis and diversion of carbon utilization for acetyl-CoA from the TCA cycle could be significant drivers of increased lipid production in oil palm. The results suggest more broadly that concerted changes in the glycolytic pathway enzyme activities, as discussed earlier, combined with changes to expression or activities of certain TCA cycle enzymes, can simultaneously direct flux towards the two key building blocks of lipid, i.e. glycerol and fatty acids, as a an approach for increasing oil yields of oil palm plants.

### Example 9

### Genetically Modifying a Plant for Increased Oil Yield

Overview.

Genetically modified plants that exhibit an increased oil yield relative to a corresponding control plant that is not so genetically modified, based on a decrease in triose-phosphate isomerase activity and an increase in glycerol-3-phosphate dehydrogenase activity, can be generated as described in the following prophetic example.

A. Genetically modify a plant, e.g. an oil-producing plant such as jatropha, camelina, maize, rapeseed, castor, oil palm, other oil crops, or a model plant system, e.g. Arabidopsis thaliana, to cause a decrease (e.g. knock down) in triose-phosphate isomerase activity.

B Optionally also genetically modify the plant to cause a decrease in glyceraldehyde-3-phosphate dehydrogenase activity.

C. Clone glycerol-3-phosphate dehydrogenase from an appropriate source, e.g. oil palm, and optionally also clone ATP citrate lyase from an appropriate source, e.g. oil palm, into a vector, under control of a strong promoter that has the capacity to be expressed in seeds of the genetically modified plant during oil deposition.

D. Transform the genetically modified plant with the vector, e.g. by Agrobacterium-mediated transformation.

E. Obtain progeny plants, T1 seeds, and then T2 seeds for analysis.

F. Measure oil content and activities of triose-phosphate isomerase and glycerol-3-phosphate dehydrogenase activity, and optionally glyceraldehyde-3-phosphate dehydrogenase and ATP citrate lyase.

These steps are described in greater detail below based on an example including genetic modification of Arabidopsis thaliana and transformation thereof with genes derived from oil palm.

A. Genetically Modify a Plant to Cause a Decrease in Triose-Phosphate Isomerase Activity.

Alleles of triose-phosphate isomerase are cloned as follows. A pair of primers are designed based on the consensus sequence of various cloned plant triose-phosphate isomeruse genes (e.g. jatropha, camelina, Arabidopsis, maize, rapeseed, castor, etc.). Total RNA is obtained, treated with DNAase, and converted into cDNAs. The resultant cDNAs are then used as templates for PCR amplification. Exemplary PCR thermocycling conditions, in accordance with Liu et al. (12), are used as follows: (i) initial denaturation at 94° C. for 2 min; (ii) 30 cycles, each consisting of denaturation at 94° C. for 45 s, annealing at 50° C. for 1 min, and elongation at 72° C. for 2 min; and (iii) a final round of elongation for 10 min at 72° C. PCR products are separated by 1% (w/v) agarose gel electrophoresis, are purified using an extract kit, and are subjected to DNA sequence analysis. Following confirmation of sequence, a PCR fragment corresponding to the triose-phosphate isomerase gene is cloned into a vector, in an antisense orientation, between a CaMV 35S promoter and an ocs terminator in the vector. The resulting construct is then introduced into Arabidopsis thaliana plants, by an Agrobacterium-mediated transformation protocol, resulting in transgenic plants that exhibit antisense expression of the triose-phosphate isomerase gene.

The transgenic plants are grown to maturity and seeds (T2) from 10 individual plants are collected after selection of putative transformants and growth of the transformants up to T2 seeds, in accordance with Elhiti et al. (13) and Jain et al. (14). After growth for approximately 4 weeks in a growth chamber, the siliques are mature and dry, and seeds are harvested and are selected for positive transformants.

Gene confirmation in transgenic plants is carried out by PCR and northern blot analysis. Initial screening of the lines is carried out using a combination of total enzyme activity determinations and northern blot, in accordance with Jako et al. (15) and Liu et al. (16). These screens allow the selection of lines, which are progressed to the next generation until T2 seeds are obtained. T1 transformants are transplanted into soil mix and grown to maturity. The mature seeds (T2 generation) are harvested from individual plants and further analyzed. Wild-type plants are transformed with empty vector and are grown as controls.

Quantification of protein levels in transgenic plants is performed using immunodetection of triose-phosphate isomerase protein. Analysis of triosc-phosphate isomerase enzyme activity is performed to confirm decreased activity. Relative quantification of the target expression of the triose-phosphate isomerase gene in wild-type and transgenic lines is performed using the comparative Ct method by quantification-real time PCR, in accordance with Sienkiewicz-Porzucek et al. (17) and Schmittgen et al. (18).

As will be appreciated by one of ordinary skill in the art, completely eliminating triose-phosphate isomerase activity, and optionally glyceraldehyde-3-phosphate dehydrogenase activity, e.g. based on deletion of all copies of the corresponding genes to knock out expression, in a plant such Arabidopsis would likely result in lethality, since these activities are important for carbon metabolism generally. As will also be appreciated by one of ordinary skill, the antisense approach described above can avoid this problem because it can result in genetically modified plants that are knocked down, rather than being knocked out, with respect to these activities.

B. Optionally Also Genetically Modify the Plant to Cause a Decrease in Glyceraldehyde-3-Phosphate Dehydrogenase Activity.

Optionally, further genetic modification is conducted to cause a decrease in glyceraldehyde-3-phosphate dehydrogenase activity, similarly as described above, based on introduction of a construct resulting in antisense expression of the glyceraldehyde-3-phosphate dehydrogenase into the genetically modified Arabidopsis plants that exhibit a decrease in triose-phosphate isomerase activity.

C-F. Remaining Steps.

The resulting genetically modified Arabidopsis plants, with decreased triose-phosphate isomerase activity, and optionally decreased glyceraldehyde-3-phosphate dehydrogenase activity, are transformed for overexpression of oil palm glycerol-3-phosphate dehydrogenase, and optionally also oil palm ATP citrate lyase, and are grown side by side, in the same container, with corresponding wild-type plants, in order to minimize variables that may arise from differences in the microenvironment of the growth room, in accordance with Liu et al. (12).

To accomplish this transformation, vector construction and plant transformation are carried out as follows. BLAST is used to compare the Arabidopsis glycerol-3-phosphate dehydrogenase sequence against oil palm genome database. Fragments encoding oil palm glycerol-3-phosphate dehydrogenase are amplified with the primers designed against the oil palm coding sequences using an RT-PCR kit, in accordance with Thelen et al. (19). PCR amplifications are carried out with 40 cycles of 94° C. for 30 s, 58° C. for 90 s, and 72° C. for 90 s. The amplicons are cloned into the Gateway brand entry vector pCR/GW/TOPO (Invitrogen, USA) and subsequently are transferred into pK2GW7 (for sense transformation) or pK2WG7 (for antisense transformation) vectors armed with the 35S promoter and the 35S terminators, in accordance with Liu et al. (12). The process is repeated for the ATP citrate lyase gene. Double targeted genes are constructed using the same method. The construct is transformed using the Agrobacterium-mediated floral dip method, in accordance with Zhang et al. (20). The construct is then inserted into the transgenic plant with decreased triose-phosphate isomerase activity, and optionally decreased glyceraldehyde-3-phosphate dehydrogenase activity, using Agrobacterium, in accordance with Bhalla et al. (21). Wild-type plants are transformed with empty vectors as controls.

transgenic plants are generated, putative transformants are selected, plants are grown until maturity, T2 seeds are obtained from 10 individual plants, and lipid components thereof are analyzed. After plants are grown for approximately 4 weeks in a growth chamber, the siliques are mature and dry, and seeds are harvested and are selected for positive transformants.

Transgenic lines are analyzed and confirmed. PCR is used to detect expression levels of the oil palm glycerol-3-phosphate dehydrogenase and ATP citrate lyase transgenes in transgenic Arabidopsis plants for transgenic line confirmation.

Gene expression analyses are carried out by qRT-PCR and/or microarray analysis, for example as described by Elhiti et al. (13). Expression studies are conducted for genes involved in glycolysis, TCA cycle, fatty acid metabolism and representative transcription factors regulating oil biosynthesis in the seeds. Analyses are performed on three biological replicates using samples from different plants. Microarray analyses are performed with wild-type and transgenic plants overexpressing glycerol-3-phosphate dehydrogenase and ATP citrate lyase. Seeds are harvested from 20-day-old Arabidopsis plants after germination and frozen immediately in liquid nitrogen. Total RNA is extracted using the RNeasy plant mini kit (Qiagen). Spectrophotometric analyses of the isolated total RNA are carried out at 260 nm and 280 nm to determine sample concentration and purity. Regulated genes are identified with a stringent significance threshold, namely a mean >1.5-fold change (overexpressing transgenic treatment relative to wild-type controls) and a P value ≦0.01, based on at least three out of four replicates.

Phenotypes of transgenic plants seeds are analyzed by microscopy. Mature transgenic seeds of plants overexpressing glycerol-3-phosphate dehydrogenase, and optionally ATP citrate lyase, and seeds of wild-type controls are photographed under a dissection microscope, are weighed, and are measured with respect to size. For transmission electron microscopy, seeds (28 DAP) are fixed with 3% glutaraldehyde in 2.5 mM cacodylate buffer supplemented with 5 mM CaCl₂ (pH 7.0) overnight at 4° C. Tissues are then post-fixed in a solution containing 2% OsO₄ and 0.8% KFe(CN), overnight at 4° C., rinsed three times with distilled water, stained overnight with 0.5% uranyl acetate, and finally dehydrated using ethanol and examined using transmission electron microscopy, in accordance with Elhiti et al. (13) and Graham et al. (22). Light microscopy analyses are performed as well.

Lipids of T2 seeds and wild-type seeds are analyzed. Total oil content in T2 seeds and wild-type seeds is determined. Dry Arabidopsis seeds (50 mg) are weighed accurately and are homogenized using a pestle and mortar in liquid nitrogen. Analyses of fatty acids profile are conducted using gas chromatography-mass spectrometry (GC-MS). The relative peak area is used to calculate the amount of fatty acids. Total monoacylglycerols, diacylglycerols, and triacylglycerols are determined using gas chromatography-flame ionization detector (GC-FID).

The approaches described above can also be applied to other plants, e.g. an oil-producing plant such as jatropha, camelina, maize, rapeseed, castor, oil palm, and other oil crops based on these and other methods for genetic modification that are known in the art. The approaches described above can also be applied with respect to glycerol-3-phosphate dehydrogenase and ATP citrate lyase genes from other appropriate sources, e.g. such genes from plants, microbes, or other organisms as identified in screens for corresponding enzyme activities, and can also be applied with respect to other genes, e.g. malate dehydrogenase, fructose-1,6-bisphosphate aldolase, pyruvate kinase, and aconitase, as also disclosed herein.

### Example 10

### Genetically Modifying a Microbe for Increased Oil Yield

Decreased Expression of Triose-Phosphate Isomerase and Overexpression of Glycerol-3-Phosphate Dehydrogenase.

The functional effect on decreased expression of triose-phosphate isomerase and overexpression of glycerol-3-phosphate dehydrogenase in microbes can be observed by conducting a complementation study in a microbe, such as yeast (Saccharomyces cerevisiae), corresponding to a knock down of triose-phosphate isomerase to which the glycerol-3-phosphate dehydrogenase gene of oil palm has been added for overexpression thereof, as described in the following prophetic example.

Transformation of Oil Palm Genes in Yeast.

Oil palm glycerol-3-phosphate dehydrogenase is cloned in an appropriate vector, e.g. a yeast expression vector with 2 micron to allow 100-200 copies of the gene, and later transformed into a Saccharomyces cerevisiae strain knocked down for triose-phosphate isomerase. Positive yeast transformants are selected on selection media, e.g. SC—minimal medium minus uracil. Expression of oil palm glycerol-3-phosphate dehydrogenase in a strain knocked down for triose-phosphate isomerase is analyzed by immunoblot assay to detect the presence of the glycerol-3-phosphate dehydrogenase gene. Identified metabolites and lipid profiling assays are performed to examine the effects of overexpression of glycerol-3-phosphate dehydrogenase in the strain knocked down for triose-phosphate isomerase. Methods and techniques for gene isolation, cloning, and transformation are described, for example by Sambrook et al. (23) and Gietz et al. (24).

Quantification of Proteins.

Proteins of the transformed yeast strain are obtained by total protein extraction, are quantified by Bradford assay, and are measured with respect to protein expression by immunoblot analysis. Yeast cells are harvested from liquid culture by centrifugation at 5.000×g for 3 min at 4° C. using a pre-weighed 1.5-ml tube centrifuge tube. The cell pellet is re-suspended at room temperature with pre-mixed of YeastBuster brand reagent (EMD Millipore) and THP solution by pipetting or gentle vortexing (5 ml YeastBuster reagent and 50 microliters 100×THP solution per gram wet cell paste). Renzonase (R) nuclease (Sigma Aldrich) is then added into the mixture to reduce viscosity. The cell suspension is incubated on a rotating mixer at a slow setting for 15-20 min at room temperature. The insoluble cell debris is removed by centrifugation at 16,000×g for 20 min at 4° C. The supernatant is then transferred to fresh tube and stored at −20° C. prior to analysis. Determination of protein concentration is assayed using Bradford reagent. Immunoblot blot analysis on the recombinant protein is performed using specific gene antibodies, anti-His tag, and anti-V5 epitope.

Quenching and Extraction of Intracellular Metabolites in Yeast.

Samples are harvested at different stages of growth and centrifuged for 2 min at 7500 rpm, 4° C. The supernatant is discarded and the cell pellet is re-suspended with 5 mL fresh medium. The quenching method is adapted from Castrillo et al. (7). In accordance with the method, 10 mL of 60% (v/v) buffered methanol:tricine (10 mM, pH 7.4) at −40° C. is added to sample, the mixture is quickly vortexed (about 1 s) and is incubated at −50° C. for about 5 min. Supernatant is then separated by centrifugation (7500 rpm, 5 min, −9° C., adaptors pre-cooled at −40° C.). Extraction is then carried out by a method using buffered ethanol:ammonium acetate (10 mM, pH 7.5) as adapted from Ewald et al. (8). Tubes containing 5 mL of 75% (v/v) buffered ethanol:ammonium acetate (10 mM, pH 7.5) and 0.1 mg/mL ribitol are pre-heated in a water bath at 80° C. Samples are added to the boiling buffered ethanol and the mixture is immediately placed in water at 80° C. for 3 min. Samples are then centrifuged for 2 min at 7500 rpm to remove biomass debris. Samples are filtered through a 0.2 micron filter and freeze-dried to dryness. Samples are then prepared for analysis by any of various standard approaches by reconstituting the samples in a solvent system appropriate for the approach.

Quantitation of Selected Metabolites.

The levels of one or more targeted metabolites in the yeast transformants are determined by, for example, LC-MS, GC-MS, or simple HPLC analysis using reversed-phase or amide columns and a solvent gradient from ammonium formate (pH 9.0) to acetonitrile. A calibration curve is prepared to accurately determine sample concentration using authentic standards of the targeted compounds and determining peak area of at least five different concentrations between 0.1 mg/L to 1.0 mg/L.

Total Lipid Content and Lipid Profiling.

Determination of the total lipid content is conducted by extracting lipid content using various extraction solvents e.g. hexane and chloroform/methanol/water. Changes in levels of total lipid content are compared between control and transformants with a decreased triose-phosphate isomerase activity and an over-expressed glycerol-3-phosphate dehydrogenase activity by determining the dry weight of the extract.

Changes in levels of monoacylglycerols, diacylglycerols, triacylglycerols, phospholipids, and fatty acids between control and transformants with a decreased triose-phosphate isomerase activity and an over-expressed glycerol-3-phosphate dehydrogenase activity are determined by, for example, LC-MS, GC-MS, or GC-FID.

Decreased Expression of Glyceraldehyde-3-Phosphate Dehydrogenase Activity and Overexpression of Glycerol-3-Phosphate Dehydrogenase Activity.

The functional effect on decreased expression of glyceraldehyde-3-phosphate dehydrogenase activity and overexpression of glycerol-3-phosphate dehydrogenase in microbes can also be observed by conducting a complementation study in a microbe, such as yeast (Saccharomyces cerevisiae), corresponding to a knock down of glyceraldehyde-3-phosphate dehydrogenase activity to which the glycerol-3-phosphate dehydrogenase gene of oil palm has been added, as described in the following prophetic example. The glycerol-3-phosphate dehydrogenase gene is cloned into suitable yeast expression vector, e.g. a vector with 2 micron. The yeast strain that is knocked down for glyceraldehyde-3-phosphate dehydrogenase activity is then transformed with the vector. Selection of positive transformants is made on minimal medium minus uracil. The presence of glycerol-3-phosphate dehydrogenase protein is detected via immunoblot blot assay with a specific antibody. Increases in lipid content and changes or flux in levels of the metabolites are measured quantitatively by, for example, LC-MS, GC-MS or simple HPLC analysis.

Decreased Expression of Triose-Phosphate Isomerase and Overexpression of Glycerol-3-Phosphate Dehydrogenase and Fructose-1,6-Bisphosphate Aldolase.

Double overexpression of glycerol-3-phosphate dehydrogenase and fructose-1,6-bisphosphate aldolase in combination with knock down of triose-phosphate isomerase in yeast strains can lead to higher lipid content as compared to single overexpression of either glycerol-3-phosphate dehydrogenase or fructose-1,6-bisphosphate aidolase in the same strains, as described in the following prophetic example. Both oil palm glycerol-3-phosphate dehydrogenase and fructose-1,6-bisphosphate aldolase are subcloned into a yeast expression vector, either two different vectors with two different selection markers, e.g. uracil or tryptophan markers, or into one vector with two cloning sites, e.g. pESC-HIS. The genes are then transformed into yeast strains knocked down for triose-phosphate isomerase and positive transformants are selected using selection minimal medium. Recombinant proteins corresponding to glycerol-3-phosphate dehydrogenase and/or fructose-1,6-bisphosphate aldolase are detected using immunoblot assay to confirm the presence of the genes. Lipid and metabolite profiling are measured quantitatively by, for example, LC-MS, GC-MS, or simple HPLC analysis.

### REFERENCES

1. He C Y, Zhan J G, Duan A G, Yin J Y and Zhou D S. Comparison of Methods for Protein Extraction from Pine Needles. Forestry Studies in China, 7: 20-23 (2005).
2. Asemota O and Shah F H. Detection of Mesocarp Oleoyl-Thioesterase Gene of the South American Oil Palm Elaeis oleifera by Reverse Transcriptase Polymerase Chain Reaction. African Journal of Biotechnology, 3: 595-598 (2004).
3. Agilent. One-Color Microarray-Based Gene Expression Analysis, Low Input Quick Amp Labeling Protocol. Agilent Technologies, Version 6.0 (December 2009).
4. Fodor A, Tickle T, and Richardson C. Towards the Uniform Distribution of Null P Values on Affymetrix Microarrays. Genome Biology, 8: R69 (2007).
5. Ye J, Fang L, Zheng H, Zhang Y, Chen J, Zhang Z, Wang J, Li S, Li R, Bolund L and Wang J. WEGO: A Web Tool for Plotting G O Annotations. Nucleic Acids Research, 34 (Web Server issue): W293-W297 (2006).
6. Wang Z Y, Fang B P, Chen J Y, Zhang X J, Luo Z X, Huang L F, Chen X L and Li Y J. De Novo Assembly and Characterization of Root Transcriptome Using Illumina Paired-End Sequencing and Development of cSSR Markers in Sweet Potato (Ipomoea batatas). BMC Genomics, 11:726 (2010).
7. Castrillo J I, Hayes A, Mohammed S, Gaskell S J and Oliver S G. An Optimized Protocol for Metabolome Analysis in Yeast Using Direct Infusion Electrospray Mass Spectrometry. Phytochemistry, 62: 929-937 (2003).
8. Ewald J C, Heux S and Zamboni N. High-Throughput Quantitative Mctabolomics: Workflow for Cultivation, Quenching, and Analysis of Yeast in a Multiwell Format. Anal. Chem., 81: 3623-3629 (2009).
9. Rius S P, Casati P, Iglesias A A and Gomez-Casati D F. Characterization of Arabidopsis Lines Deficient in GAPC-1, a Cytosolic NAD-Dependent Glyceraldehyde-3-Phosphate Dehydrogenase. Plant Physiology, 148: 1655-1667 (2008).
10. Wang Z X, Zhugea J, Fang H Y and Prior P A. Glycerol Production by Microbial Fermentation: A Review. Biotechnol. Advances, 19: 201-223 (2001).
11. Remize F, Barnavon L, and Dequin S. Glycerol Export and Glycerol-3-Phosphate Dehydrogenase, but not Glycerol Phosphatase, Are Rate Limiting for Glycerol Production in Saccharomyces cerevisiae. Metabolic Eng., 3: 301-312 (2001).
12. Liu J, Hua W, Yang H L, Zhan G M, Li R J, Deng L B, Wang X F, Liu G H, and Wang H Z. The BnGRF2 Gene (GRF2-Like Gene from Brassica napus) Enhances Seed Oil Production Through Regulating Cell Number and Plant Photosynthesis. J Exp Bot, 63: 3727-3740 (2012).
13. Elhiti M, Yang C, Chan A, Dumin D C, Belmonte M F, Ayele B T, Tahir M, and Stasolla C. Altered Seed Oil and Glucosinolate Levels in Transgenic Plants Overexpressing the Brassica napus SHOOTMERISTEMLESS Gene. J. Exp. Bot., 63: 4447-4461 (2012).
14. Jain R K, Coffey M, Lai K, Kumar A, MacKenzie S L. Enhancement of Seed Oil Content by Expression of Glycerol-3-Phosphate Acyltransferase Genes. Biochemical Society Transactions. 28: 958-961 (2000).
15. Jako C, Kumar A, Wei Y, Zou J, Barton D L, Giblin E M, Covello P S, and Taylor D C. Seed-Specific Over-Expression of an Arabidopsis cDNA Encoding a Diacylglycerol Acyltransferase Enhances Seed Oil Content and Seed Weight. Plant Physiology, 126: 861-874 (2001).
16. Liu J, Hua W, Zhan G, Wei F, Wang X, Liu G, and Wang H. Increasing Seed Mass and Oil Content in Ttransgenic Arabidopsis by the Overexpression of wri1-Like Gene from Brassica napus. Plant Physiology & Biochemistry: PPB/Societe Francaise de Physiologie Vegetale, 48: 9-15 (2010).
17. Sienkiewicz-Porzucek A, Sulpice R, Osorio S, Krahnert I, Leisse A, Urbanczyk-Wochniak E, Hodges M, Fernie A R, and Nunes-Nesi A. Mild Reductions in Mitochondrial NAD-Dependent Isocitrate Dehydrogenase Activity Result in Altered Nitrate Assimilation and Pigmentation but Do Not Impact Growth. Mol Plant, 3: 156-173 (2010).
18. Schmittgen T D, and Livak K J. Analyzing Real-Time PCR Data by the Comparative C T Method. Nat Protocols, 3: 1101-1108 (2008).
19. Thelen J J, Ohlrogge J B. Both Antisense and Sense Expression of Biotin Carboxyl Carrier Protein Isoform 2 Inactivates the Plastid Acetyl-coenzyme A Carboxylase in Arabidopsis thaliana. The Plant Journal for Cell and Molecular Biology, 32: 419-431 (2002).
20. Zhang X, Henriques R, Lin S-S, Niu Q-W, and Chua N-H. Agrobacterium-mediated transformation of Arabidopsis thaliana using the floral dip method. Nat Protocols, 1: 641-646 (2006).
21. Bhalla P L, Singh M B. Agrobacterium-Mediated Transformation of Brassica napus and Brassica oleracea. Nat. Protocols, 3: 181-189 (2008).
22. Graham L, and Orenstein J M. Processing Tissue and Cells for Transmission Electron Microscopy in Diagnostic Pathology and Research. Nat. Protocols. 2: 2439-2450 (2007).
23. Sambrook et al. Molecular Cloning: A Laboratory Manual (3rd edition). Cold Spring Harbor Laboratory Press, Cold Spring Harbor, N.Y. (2001).
24. Gietz et al. Studies on the Transformation of Intact Yeast Cells by LiAc/SS-DNA/PEG procedure. Yeast, 11: 355-360 (1995).

### INDUSTRIAL APPLICABILITY

The methods and kits disclosed herein are useful for genetically modifying a plant or microbe and for increasing oil yields in plants and microbes, and thus for improving commercial production of oils derived from plants and microbes.

1. A method for obtaining a genetically modified plant, wherein the plant exhibits an increased oil yield relative to a corresponding control plant that is not so genetically modified, comprising:
genetically modifying a plant progenitor cell to cause a decrease in triose-phosphate isomerase activity and an increase in glycerol-3-phosphate dehydrogenase activity; and
culturing the genetically modified plant progenitor cell to obtain the genetically modified plant.

2. The method of claim 1, wherein the genetic modification further causes at least one of an increase in malate dehydrogenase activity and an increase in ATP citrate lyase activity.

3. The method of claim 2, wherein the genetic modification causes both the increase in malate dehydrogenase activity and the increase in ATP citrate lyase activity.

4. The method of claim 1, 2, or 3, wherein the genetic modification further causes at least one of a decrease in glyceraldehyde-3-phosphate dehydrogenase activity, an increase in fructose-1,6-bisphosphate aldolase activity, an increase in pyruvate kinase activity, and an increase in aconitase activity.

5. The method of claim 4, wherein the genetic modification causes all four of the decrease in glyceraldehyde-3-phosphate dehydrogenase activity, the increase in fructose-1,6-bisphosphate aldolase activity, the increase in pyruvate kinase activity, and the increase in aconitase activity.

6. The method of any of claims 1 to 5, wherein at least one of the decreases in activity and at least one of the increases in activity contribute to confer the increased oil yield of the genetically modified plant.

7. The method of any of claims 1 to 6, wherein all of the decreases in activity and the increases in activity occur in at least one oil-producing organ or tissue of the genetically modified plant, wherein the oil-producing organ or tissue is selected from the group consisting of fruit, mesocarp, kernel, and seed.

8. The method of any of claims 1 to 7, wherein all of the decreases in activity and the increases in activity occur in at least one oil-producing organ or tissue of the genetically modified plant at least during onset of oil deposition in the oil-producing organ or tissue.

9. The method of any of claims 1 to 8, further comprising selecting the genetically modified plant based on at least one of the decreases in activity and the increases activity.

10. The method of any of claims 1 to 9, wherein at least one of the decreases in activity is based on a technique selected from the group consisting of mutagenesis, RNAi, expression of siRNA, gene silencing, homologous recombination, disruption of a regulatory sequence, partial gene deletion, and full gene deletion.

11. The method of any of claims 1 to 10, wherein at least one of the increases in activity is based on a technique selected from the group consisting of transformation, Agrobacterium-mediated transformation, viral transformation, particle bombardment, introduction of recombinant DNA, introduction of a plasmid, and introduction of an artificial chromosome.

12. The method of any of claims 1 to 11, wherein at least one of the decreases in activity is based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene.

13. The method of any of claims 1 to 12, wherein at least one of the increases in activity is based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene.

14. The method of any of claims 1 to 13, wherein the oil yield of the genetically modified plant is increased by at least 10% relative to the corresponding control plant.

15. The method of any of claims 1 to 14, wherein the plant progenitor cell is selected from the group consisting of an isolated cell, a cell of a plant leaf, a cell of a plant flower, and a cell of a plant embryo.

16. The method of any of claims 1 to 15, wherein the genetically modified plant is an oil crop selected from the group consisting of oil palm, olive, coconut palm, soybean, sunflower, rapeseed, field mustard, canola, peanut, corn, cotton, safflower, castor, jatropha, and camelina.

17. A method of producing oil from a genetically modified plant that exhibits an increased oil yield relative to a corresponding control plant that is not so genetically modified, comprising:
obtaining a genetically modified plant by the method of any of claims 1 to 16; and
extracting oil from an oil-producing organ or tissue of the genetically modified plant.

18. A genetically modified plant that exhibits an increased oil yield relative to a corresponding control plant that is not so genetically modified, wherein the genetically modified plant is obtained according to any of claims 1 to 16.

19. A method for obtaining a genetically modified microbe, wherein the microbe exhibits an increased oil yield relative to a corresponding control microbe that is not so genetically modified, comprising:
genetically modifying a microbial cell to cause a decrease in triose-phosphate isomerase activity and an increase in glycerol-3-phosphate dehydrogenase activity; and
culturing the microbial cell to obtain the genetically modified microbe.

20. The method of claim 19, wherein the genetic modification further causes at least one of an increase in malate dehydrogenase activity and an increase in ATP citrate lyase activity.

21. The method of claim 20, wherein the genetic modification causes both the increase in malate dehydrogenase activity and the increase in ATP citrate lyase activity.

22. The method of claim 19, 20, or 21, wherein the genetic modification further causes at least one of a decrease in glyceraldehyde-3-phosphate dehydrogenase activity, an increase in fructose-1,6-bisphosphate aldolase activity, an increase in pyruvate kinase activity, and an increase in aconitase activity.

23. The method of claim 22, wherein the genetic modification causes all four of the decrease in glyceraldehyde-3-phosphate dehydrogenase activity, the increase in fructose-1,6-bisphosphate aldolase activity, the increase in pyruvate kinase activity, and the increase in aconitase activity.

24. The method of any of claims 19 to 23, wherein at least one of the decreases in activity and at least one of the increases in activity contribute to confer the increased oil yield of the genetically modified microbe.

25. The method of any of claims 19 to 24, further comprising selecting the genetically modified microbe based on at least one of the decreases in activity and the increases in activity.

26. The method of any of claims 19 to 25, wherein at least one of the decreases in activity is based on a technique selected from the group consisting of mutagenesis, RNAi, expression of siRNA, gene silencing, homologous recombination, disruption of a regulatory sequence, partial gene deletion, and full gene deletion.

27. The method of any of claims 19 to 26, wherein at least one of the increases in activity is based on a technique selected from the group consisting of transformation, electroporation, transduction, introduction of recombinant DNA, introduction of a plasmid, and introduction of an artificial chromosome.

28. The method of any of claims 19 to 27, wherein at least one of the decreases in activity is based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene.

29. The method of any of claims 19 to 28, wherein at least one of the increases in activity is based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene.

30. The method of any of claims 19 to 29, wherein the oil yield of the genetically modified microbe is increased by at least 10% relative to the corresponding control microbe.

31. The method of any of claims 19 to 30, wherein the genetically modified microbe is selected from the group consisting of an oleaginous microbe, an oleaginous bacterium, an oleaginous actinomycetes, an oleaginous Mycobacterium, an oleaginous Streptomyces, an oleaginous Rhodococcus, an oleaginous Nocardia, an oleaginous fungus, an oleaginous yeast, and an oleaginous Mortierella.

32. A method of producing oil from a genetically modified microbe that exhibits an increased oil yield relative to a corresponding control microbe that is not so genetically modified comprising:
obtaining the genetically modified microbe by the method of any of claims 19 to 31; and
extracting oil from the genetically modified microbe.

33. A genetically modified microbe that exhibits an increased oil yield relative to a corresponding control microbe that is not so genetically modified, wherein the genetically modified microbe is obtained according to any of claims 19 to 31.

34. A method for increasing oil yield, comprising genetically modifying a plant to cause, in at least one oil-producing organ or tissue of the plant, a decrease in triose-phosphate isomerase activity and an increase in glycerol-3-phosphate dehydrogenase activity, wherein:
the genetic modification is carried out across more than a single generation of the plant; and
the genetically modified plant exhibits an increased oil yield relative to a corresponding control plant that is not so genetically modified.

35. The method of claim 34, wherein the genetic modification further causes at least one of an increase in malate dehydrogenase activity and an increase in ATP citrate lyase activity.

36. The method of claim 35, wherein the genetic modification causes both the increase in malate dehydrogenase activity and the increase in ATP citrate lyase activity.

37. The method of claim 34, 35, or 36, wherein the genetic modification further causes at least one of a decrease in glyceraldehyde-3-phosphate dehydrogenase activity, an increase in fructose-1,6-bisphosphate aldolase activity, an increase in pyruvate kinase activity, and an increase in aconitase activity.

38. The method of claim 37, wherein the genetic modification causes all four of the decrease in glyceraldehyde-3-phosphate dehydrogenase activity, the increase in fructose-1,6-bisphosphate aldolase activity, the increase in pyruvate kinase activity, and the increase in aconitase activity.

39. The method of any of claims 34 to 38, wherein at least one of the decreases in activity and at least one of the increases in activity contribute to confer the increased oil yield of the genetically modified plant.

40. The method of any of claims 34 to 39, wherein all of the decreases in activity and the increases in activity occur in the at least one oil-producing organ or tissue of the genetically modified plant, and the oil-producing organ or tissue is selected from the group consisting of fruit, mesocarp, kernel, and seed.

41. The method of any of claims 34 to 40, wherein all of the decreases in activity and the increases in activity occur in the at least one oil-producing organ or tissue of the genetically modified plant at least during onset of oil deposition in the oil-producing organ or tissue.

42. The method of any of claims 34 to 41, further comprising selecting the genetically modified plant based on at least one of the decreases in activity and the increases activity.

43. The method of any of claims 34 to 42, wherein at least one of the decreases in activity is based on a technique selected from the group consisting of mutagenesis, RNAi, expression of siRNA, gene silencing, homologous recombination, disruption of a regulatory sequence, partial gene deletion, and full gene deletion.

44. The method of any of claims 34 to 43, wherein at least one of the increases in activity is based on a technique selected from the group consisting of transformation, Agrobacterium-mediated transformation, viral transformation, particle bombardment, introduction of recombinant DNA, introduction of a plasmid, and introduction of an artificial chromosome.

45. The method of any of claims 34 to 44, wherein at least one of the decreases in activity is based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene.

46. The method of any of claims 34 to 45, wherein at least one of the increases in activity is based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene.

47. The method of any of claims 34 to 46, wherein the oil yield of the genetically modified plant is increased by at least 10% relative to the corresponding control plant.

48. The method of any of claims 34 to 47, wherein the genetically modified plant is an oil crop selected from the group consisting of oil palm, olive, coconut palm, soybean, sunflower, rapeseed, field mustard, canola, peanut, corn, cotton, safflower, castor, jatropha, and camelina.

49. A method of producing oil, comprising:
obtaining a genetically modified plant by the method of any of claims 34 to 48; and
extracting oil from the oil-producing organ or tissue of the genetically modified plant.

50. A genetically modified plant that exhibits an increased oil yield relative to a corresponding control plant that is not so genetically modified, wherein the genetically modified plant is obtained according to any of claims 34 to 48.

51. A method for increasing oil yield, comprising genetically modifying a microbe to cause a decrease in triose-phosphate isomerase activity and an increase in glycerol-3-phosphate dehydrogenase activity in the microbe, wherein:
the genetic modification is carried out across more than a single generation of the microbe; and
the genetically modified microbe exhibits an increased oil yield relative to a corresponding control microbe that is not so genetically modified.

52. The method of claim 51, wherein the genetic modification further causes at least one of an increase in malate dehydrogenase activity and an increase in ATP citrate lyase activity.

53. The method of claim 52, wherein the genetic modification causes both the increase in malate dehydrogenase activity and the increase in ATP citrate lyase activity.

54. The method of claim 51, 52, or 53, wherein the genetic modification further causes at least one of a decrease in glyceraldehyde-3-phosphate dehydrogenase activity, an increase in fructose-1,6-bisphosphate aldolase activity, an increase in pyruvate kinase activity, and an increase in aconitase activity.

55. The method of claim 54, wherein the genetic modification causes all four of the decrease in glyceraldehyde-3-phosphate dehydrogenase activity, the increase in fructose-1,6-bisphosphate aldolase activity, the increase in pyruvate kinase activity, and the increase in aconitase activity.

56. The method of any of claims 51 to 55, wherein at least one of the decreases in activity and at least one of the increases in activity contribute to confer the increased oil yield of the genetically modified microbe.

57. The method of any of claims 51 to 56, further comprising selecting the genetically modified microbe based on at least one of the decreases in activity and the increases in activity.

58. The method of any of claims 51 to 57, wherein at least one of the decreases in activity is based on a technique selected from the group consisting of mutagenesis, RNAi, expression of siRNA, gene silencing, homologous recombination, disruption of a regulatory sequence, partial gene deletion, and full gene deletion.

59. The method of any of claims 51 to 58, wherein at least one of the increases in activity is based on a technique selected from the group consisting of transformation, electroporation, transduction, introduction of recombinant DNA, introduction of a plasmid, and introduction of an artificial chromosome.

60. The method of any of claims 51 to 59, wherein at least one of the decreases in activity is based on an effect selected from the group consisting of a decrease in specific activity of a corresponding enzyme, a decrease in copy number of a corresponding gene, a deleterious mutation in a corresponding gene, a deleterious modification of a corresponding enzyme, and a decrease in transcription of a corresponding gene.

61. The method of any of claims 51 to 60, wherein at least one of the increases in activity is based on an effect selected from the group consisting of an increase in specific activity of a corresponding enzyme, an increase in copy number of a corresponding gene, an advantageous mutation in a corresponding gene, an advantageous modification of a corresponding enzyme, and an increase in transcription of a corresponding gene.

62. The method of any of claims 51 to 61, wherein the oil yield of the genetically modified microbe is increased by at least 10% relative to the corresponding control microbe.

63. The method of any of claims 51 to 62, wherein the genetically modified microbe is selected from the group consisting of an oleaginous microbe, an oleaginous bacterium, an oleaginous actinomycetes, an oleaginous Mycobaclerium, an oleaginous Streptomyces, an oleaginous Rhodococcus, an oleaginous Arocardia, an oleaginous fungus, an oleaginous yeast, and an oleaginous Mortierella.

64. A method of producing oil, comprising:
obtaining a genetically modified microbe by the method of any of claims 51 to 63; and
extracting oil from the genetically modified microbe.

65. A genetically modified microbe that exhibits an increased oil yield relative to a corresponding control microbe that is not so genetically modified, wherein the genetically modified microbe is obtained according to any of claims 51 to 63.


SEQUENCE LISTING (9 sequences)

SEQ ID NO: 1 - protein, 254 aa, Elaeis guineensis
MGRRFFVGGNWKCNGTSEEVKKIVSTLNDGQVPSSDVVEVVISPPYVFLPLVKSSLRPDFHVAAQNCWVKKGGAFTGEVSAEMLKNIDIHWVILGHSERRLLLGESNEFVGDKVAYALSQGLKVIACVGETLEQRESGATMDVVAAQTKAIAERISDWTNVVVAYEPVWAIGTGKVATPAQAQEVHFALRKWLETNVSAQVAESTRIIYGGSVNGANCKELAAQPDVDGFLVGGASLKAEFVDIIKSATVKSSA

SEQ ID NO: 2 - protein, 307 aa, Elaeis guineensis
MAAASSLVPQFSGLAVARERAPSLSSILFFHGASCQRRPFSTRRAPRGVVAMAGTGKFFVGGNWKCNGMKDSISKLVADLNDAKMENDVDVAVAPPFIYIDQVNNSLTDRIDISAQNCWVGKGGAFTGEISAEQLIDMGCKWVILGHSERRHIIGEDDQFIGKKAAYALSQNLKVIACIGEKLEEREAGKTFDVCFQQMKAFADSITSWEDVVIAYEPVWAIGTGKVATPEQAQEVHGAVRDWLKKNISEEVASSTRIIYGGSVNGSNCSELAKQEDIDGFLVGGASLKGPEFGTIVNSVTSKKVAA

SEQ ID NO: 3 - protein, 462 aa, Elaeis guineensis
MVGSIEAKSLQSNGSVHHIGLNLEEKLDEFRRLLGKSEKDPLRIVSVGAGAWGSVFAALLQESYGGFRDKFQIRIWRRAGRAVDRETAEHLFEVINSREDILRRLIRRCAYLKYVEARLGDRTLYADEILKDGFCLNMVDTPLCPLKVVTNLQEAVWDADIVVNGLPSTETREVFEEISKYWKERITVPIIISLSKGIETALEPVPHIITPTKMIHQATGVPIDNVLYLGGPNIAAEIYNKEYANARICGAAKWRKPLAKFLRQPHFIVWDNSDLVTHEVMGGLKNVYAIGAGMVAALTNESATSKSVYFAHCTSEMIFITHLLAEEPEKLAGPLLADTYVTLLKGRNAWYGQMLAKGEINRDMGDSISGKGMIQGVSAVGAFYQLLSQSSLSILPSEEKKPVAPVESCPILKTLYKILITREQSTQAILQALRDETLNDPRDRIEIAQSHAFYRPSLLGQP

SEQ ID NO: 4 - protein, 300 aa, Elaeis guineensis
MERSAEASRRIARISSHLQPPSSQMEEISLLRRSNCRSKGGAPGFKVAILGAAGGIGQPLALLMKMNPLVSVLHLYDVVNTPGATADISHMDTGAVVRGFLGQPQLESALIGMGLVIIPAGVPRKPGMTRDDLFNINAGIVRTLCEGIAKWCPNAIVNVISNPLNSTVPIAAEVFKQAGTYDPKHLLGVTTLDVVRANTFVAEVLGINPREVSVPVVGGHAGVTILPLLSQVNPPYSFTTEEIRYLTDRIQNGGTEVVEAKAGAGSATLSMAYAAAKFADACLRGLRGDAGIVECSLLLL

SEQ ID NO: 5 - protein, 608 aa, Elaeis guineensis
MATGQLFSRQTQALFYNYKQLPIQRMLDFDFLCGRETPSVAGVINPGSEGFQKLFFGQEEIAIPVHSSIEAACNAHPTADVFINFASFRSAAASTMSALRQPTIKVVAIIAEGVPESDTKQLIAYARANNKVVIGPATVGGIQAGAFKIGDTAGTIDNIIHCKLYRPGSVGFVSKSGGMSNELYNTIARVTDGIYEGIAIGGDVFPGSTLSDHVLRFNNIPQVKMMVVLGELGGRDEYSLVEALKEGRVHKPVVAWVSGTCARLFKSEVQFGHAGAKSGGELESAQAKNEALREAGAVVPTSYEALEGAIKETFQKLVEEGKIAPVSEVKPPQIPEDLNIAIKSGKVRAPTHIISTISDDRGDEPCYGGVPMSTIVEQGYGVGDVISLLWFKRSLPRYCTKFIEICIMLCADHGPCVSGAHNSIVTARAGKDLVSSLVSGLLTIGPRFGGAIDDAARYFKDAYDRGLTPYEFVEGMKKKGIRVPGIGHRIKSRDNRDKRVQLLQQYAHTHFPSVKYMEYAVQVETYTLSKANNLVLNVDGAIGSLFLDLLSGSGMFNKQEIDEIIEIGYLNGLFVLARSIGLIGHTFDQKRLKQPLYRHPWEDVLYTK

SEQ ID NO: 6 - protein, 206 aa, Elaeis guineensis
MFVVGVNEKEYKSDINVVSNASCTTNCLAPLAKVIHDKFGIVEGLMTTVHSITATQKTVDGPSSKDWRGGRAASFNIIPSSTGAAKAVGKVLPALNGKLTGMAFRVPTVDVSVVDLTVRLEKSATYDQIKDAIKEESEGKMKGILGYVDEDLVSTDFLGDSRSSIFDAKAGIALNGNFVKLVSWYDNEWGYSSRVVDLIRHIHGTQ

SEQ ID NO: 7 - protein, 388 aa, Elaeis guineensis
MASATLLKSSFLPKKSEWLNARPSSTSQPMAVSFTVRAGAYADELVKTAKTVASPGRGILAMDESNATCGKRLASIGLENTEANRQAYRTLLITAPGLGQYVSGAILFEETLYQSTTDGKKMVDVLVSQNIMPGIKVDKGLVPLAGSNDESWCQGLDGLASRCAAYYQQGARFAKWRTVVSIPNGPSALAVKEAAWGLARYAAIAQDNGLVPIVEPEILLDGDHGIERTFEVAQKVWAEVFFYMAENNVMYEGILLKPSMVTPGAECKERATPEQVAEYTLKLLHRRIPPAVPGIMFLSGGQSEVEATLNLNAMNQSPHPWHVSFSFARALQNTCLKTWGGRPENVKAAQDALLIRAKANSLAQLGKYTGEGESAEAKEGMYVKNYSY

SEQ ID NO: 8 - protein, 664 aa, Elaeis guineensis
SIWTESAQRSLCIFFQGFSLLTHSFRWCSRFHRVRRVPSPSYDRTDTDKRTQKNPASSPSSSFLNSPRLLFLILETWMATATEIGSLAARIRPSSGGDQAGLASSRRSLDRSLLRAGTVCRAPQPARWGLVVRSLKATERSGRAVDVVGTSNGPVSSEISNSSFALPSTEHSLEETRVVSNPRRKTKIVCTIGPSTNTREMIWKLAEAGMNVARLNMSHGDHESHQKIIDLVKEYNAQSKGNVIAIMLDTKGPEVRSGDVPQPILLKEGQEFNFTIKRGVSSEDTVSVNYDDFVNDVEVGDILLVDGGMMSLAVRSKTVDTVKCEVIDGGELKSRRHLNVRGKSATLPSITEKDWEDIKFGVDHKVDFYAVSFVKDAKVVHELKDYLKRCNADIHVIVKIESADSIQNLHSIISASDGAMVARGDLGAELPIEEVPLLQEEIINRCRSMEKPVIVATNMLESMINHPTPTRAEVSDIAIAVREGADAIMLSGETAHGKYPLKAVKVMHTVAWRTESTQSTSNNQPVLPSAVQAAICGEFSQRHMSAMFAAHATTMANTLGTPIIVFTQTGSMAILLSHYRPSSTIFAFTNEERIKQRLALYHGVLPIHMQFSNDAEETFSRAIKYLLDRKYLEKEEYVTLVQSGIHSIWRQESTHHIQVRKVQG

SEQ ID NO: 9 - protein, 995 aa, Elaeis guineensis
MMTAGALTRSSSSRLSFPLSAAAKSSSHLTAPPSSPPPSPSNPSPRSPIPPSRHSPALFSSFARYFGDWRSPLSQRAQIRYSAAVAERFERYFATMATRNAYESILTSLPKPGGGEFGKYYSLPALNDPRIDRLPYSIRILLESAIRNCDEFQVTGKDVEKILDWENTSPKQVEIPFKPARVLLQDFTGVPAVVDLACMRDAMKRLGSDSNKINPLVPVDLVIDHSVQVDVARSENAVQANMELEFHRNKERFGFLKWGSSAFHNMLVVPPGSGIVHQVNLEYLARVVFNNGGMLYPDSVVGTDSHTTMVDGLGVSGWGVGGIEAEAAMLGQPMSMVLPGVVGFKLSGKLRNGVTATDLVLTVTQMLRKHGVVGKFVEFYGEGMSELSLADRATIANMSPEYGATMGFFPVDHVTLQYLKLTGRNDDTVAMIESYLRANKLFVDYSQPQTERVYSSYLELNLEDVEPCVSGPKRPHDRVPLKVMKADWHSCLDNKVGFKGFAVPKEAQNKVAEFSFHGTPAQIKHGDVVIAAITSCTNTSNPSVMLGAALVAKKACELGLEVKPWIKTSLAPGSGVVTKYLEKSGLQKYLNQLGFHIVGYGCTTCIGNSGDLDETVSAAISENDIVAAAVLSGNRNFEGRVHPLTRANYLASPPLVVAYALAGTVDIDFETEPIGTSNDGKKVYFKDIWPSTEEIANVVQSSVLPDMFKGTYQAITKGNPMWNQLLVPSSTLYTWDPSSTYIHEPPYFKDMTMSPPGPHPVKDAYCLLNFGDSITTDHISPAGSIHKDSPAAKYLMERGVERKDFNSYGSRRGNDEVMARGTFANIRLVNKLLKGEAGPKTIHIPSGEKLSVFDAAMRYKSEEHDTVILAGAEYGSGSSRDWAAKGPMLLGVKAVIAKSFERIHRSNLVGMGIIPLCFKPGEDAETLGLTGHERYTINLPDNVSDIKPGQDVTVVTDNGKSFTCTVRFDTEVELAYYNHGGILPFVIRNLINSKH